(19) United States
(12) Patent Application Publication
(10) Pub. No.: US 2026/0167597 A1
(43) Pub. Date: Jun. 18, 2026

(54) METHOD FOR PRODUCING 2-OCTYL (METH)ACRYLATE

(71) Applicant: BASF SE, Ludwigshafen am Rhein (DE)

(72) Inventors: Andrea MISSKE, Ludwigshafen am Rhein (DE); Ortmund LANG, Ludwigshafen am Rhein (DE); Petra DECKERT, Ludwigshafen am Rhein (DE); Cornelis Hendricus DE RUITER, Ludwigshafen am Rhein (DE); Aykan AKIN, Shanghai (CN); Frank KORPJUHN, Ludwigshafen am Rhein (DE); Christoph FLECKENSTEIN, Ludwigshafen am Rhein (DE); Friederike FLEISCHHAKER, Ludwigshafen am Rhein (DE); Sabine EICHHORN, Ludwigshafen am Rhein (DE)

(21) Appl. No.: 18/712,317

(22) PCT Filed: Nov. 14, 2022
(86) PCT No.: PCT/EP2022/081719

§ 371 Date: May 22, 2024

(30) Foreign Application Priority Data
Nov. 25, 2021 (EP) 21210376.6

(51) Int. Cl. C07C 67/08; C07C 67/54; C07C 69/54

(52) U.S. Cl. CPC C07C 67/08; C07C 67/54; C07C 69/54

(57) ABSTRACT

The invention relates to a process for producing 2-octyl (meth)acrylate by reacting 2-octanol with (meth)acrylic acid in the presence of an acidic esterification catalyst, a polymerization inhibitor and the azeotroping agent cyclohexane, comprising the steps of:
providing a reactor unit (24), wherein a reactor (1) having a reactor heating element (30) is located within the reactor unit (24),
feeding 2-octanol (13), (meth)acrylic acid (14), acidic esterification catalyst (15), cyclohexane (17) and polymerization inhibitor (31) into the reactor (1),
carrying out an esterification in the reactor (1) to form a liquid reaction mixture, wherein the esterification in the reactor (1) is conducted at a bottom temperature in the range of 90 to 130° C. and at an absolute pressure in the range of 0.5 to 2.0 bar, and
a resulting reaction discharge from the reactor (1) is obtained,
wherein the resulting reaction discharge comprises at least 2-octyl (meth)acrylate, 2-octanol, (meth)acrylic acid, acidic esterification catalyst, cyclohexane, water of esterification and polymerization inhibitor, and the water of esterification formed in the esterification together with the cyclohexane azeotroping agent forms a heterogeneous azeotrope,
evaporating the heterogeneous azeotrope from the liquid reaction mixture of the reactor (1), wherein the evaporation is accomplished by the reactor heating element (30), and
removing the gaseous heterogeneous azeotrope from the reactor (1),
wherein the gaseous heterogeneous azeotrope is condensed in a condenser (5) and is then fed to a phase separator (6) and the water of esterification is separated off as the lower phase and the cyclohexane as upper phase in this phase separator (6).

The present invention relates to a process for producing 2-octyl (meth)acrylate by reacting 2-octanol with (meth)acrylic acid in the presence of an acidic esterification catalyst, a polymerization inhibitor and the azeotroping agent cyclohexane.

The term (meth)acrylic acid in this document is an abbreviation for methacrylic acid and/or acrylic acid. By analogy, 2-octyl (meth)acrylate indicates 2-octyl acrylate and/or 2-octyl methacrylate.

2-Octyl (meth)acrylate is a monomer which may be used as homo- and co-monomer in radical, cationic and anionic, and also complex-catalyzed polymerizations. It is suitable for use both in emulsion polymerizations and solvent-based polymerizations or bulk polymerizations.

Important applications are, for example, decorative coatings or technical coatings for the industrial sector and also adhesives, sealants, paper chemicals such as binders and sizing agents, leather and textile chemicals such as binders and hydrophobizing agents, rheology additives, impact modifiers for plastics, pour point depressants for oil and lubricants, printing inks or reactive thinners for UV-curing resins and systems.

Known processes for industrial-scale manufacture of 2-octyl (meth)acrylate are based predominantly on the transesterification between a (meth)acrylate of a low-carbon alcohol and 2-octanol in the presence of an alkyl titanate as transesterification catalyst and at least one polymerization inhibitor. Such a process is disclosed in WO 2013/110877 A1 (Arkema France). The disadvantage of the transesterification process is the generation of an azeotrope of the alcohol of the low-boiling (meth)acrylate and the low-boiling (meth)acrylate, for example, but not limited to, methanol/methyl methacrylate or ethanol/ethyl acrylate which has to be laboriously processed or disposed of.

WO 2013/064775 A1 (Arkema France) discloses a process for direct esterification to 2-octyl acrylate. By means of the direct esterification of acrylic acid with 2-octanol in the presence of a catalyst and a polymerization inhibitor, the 2-octyl acrylate product of value is obtained, wherein the water of esterification formed by esterification forms a heterogeneous azeotrope with the 2-octanol and is separated off by distillation in a distillation column attached to the reactor. In this case, the reactor is equipped with a stirrer and with an external heat exchanger. In a downstream purification step, the catalyst is at least partly recycled to the reactor and the 2-octyl acrylate product of value is separated.

WO 2008/046000 A1 (3M Innovative Properties Company, USA) discloses another process for the direct esterification to 2-octyl acrylate. By means of the direct esterification of acrylic acid with 2-octanol in the presence of the catalyst p-toluenesulfonic acid, the polymerization inhibitor phenothiazine and the azeotroping agent toluene, the 2-octyl acrylate product of value is obtained, wherein the water of esterification formed by the esterification forms a heterogeneous azeotrope with the toluene azeotroping agent and this heterogeneous azeotrope is separated by distillation in a Dean-Stark distillation column attached to the reactor.

These two processes described above for the direct esterification to 2-octyl acrylate have the particular disadvantage that a high energy input is required since the water of esterification formed during the esterification has to be removed from the reactor by evaporation in order to be able to separate off the evaporated water of esterification in a distillation column attached to the reactor. Therefore, the high boiling temperature of water or the high boiling temperature of the heterogeneous azeotrope “water of esterification with toluene and/or water of esterification with 2-octanol in excess” must not only be reached in the bottom of the reactor (1), but must even be exceeded, so that the required boiling temperature can be reached at the top of the distillation column.

The object was therefore to provide a process for producing 2-octyl (meth)acrylate which can be operated at a lower energy input, at a lower bottom temperature in the reactor and at moderate pressures, such as at standard pressure for example, without having to use complex apparatus. In addition, the process should achieve a better space-time yield at the same energy use than the known processes from the prior art cited above.

This object is achieved according to the present invention by a process for producing 2-octyl (meth)acrylate according to claim 1. Advantageous embodiments of the process are presented in claims 2 to 15.

The process according to the invention for producing 2-octyl (meth)acrylate by reacting 2-octanol with (meth)acrylic acid in the presence of an acidic esterification catalyst, a polymerization inhibitor and the azeotroping agent cyclohexane, comprises the steps of:
providing a reactor unit (24), wherein a reactor (1) having a reactor heating element (30) is located within the reactor unit (24),
feeding 2-octanol, (meth)acrylic acid, acidic esterification catalyst, cyclohexane and polymerization inhibitor into the one reactor (1),
carrying out an esterification in the one reactor (1) to form a liquid reaction mixture, wherein the esterification in the one reactor (1) is conducted at a bottom temperature in the range of 90 to 130° C. and at an absolute pressure in the range of 0.5 to 2.0 bar, and a resulting reaction discharge from the one reactor (1) is obtained, wherein the resulting reaction discharge comprises at least 2-octyl (meth)acrylate, 2-octanol, (meth)acrylic acid, acidic esterification catalyst, cyclohexane, water of esterification and polymerization inhibitor, and the water of esterification formed in the esterification together with the cyclohexane azeotroping agent forms a heterogeneous azeotrope,
evaporating the heterogeneous azeotrope from the liquid reaction mixture of the one reactor (1), wherein the evaporation is accomplished by the one reactor heating element (30), and
removing the gaseous heterogeneous azeotrope from the one reactor (1), wherein the gaseous heterogeneous azeotrope is condensed in a condenser (5) and is then fed to a phase separator (6) and the water of esterification is separated off as the lower phase and the cyclohexane as upper phase in this phase separator (6).

In this document, the reference numbers in brackets provide a better understanding when reading. The reference numbers in brackets are not restrictive but represent only one possible example of several possibilities for implementation.

In the following, the individual process stages I to VII are described, wherein process stages II to VII are to be considered as optional.

### Process Stage I: Esterification

The process according to the invention is based on the reactants 2-octanol and (meth)acrylic acid. In this document, (meth)acrylic acid is used to refer to a grade of (meth)acrylic acid which preferably has at least 98% by weight, more preferably at least 99.5% by weight (meth)acrylic acid, and in addition preferably has at most 0.2% by weight water and also preferably each at most 0.03% by weight acetic acid, propionic acid and isobutyric acid. Preferably, a grade of 2-octanol is used having at least 99% by weight 2-octanol, at most 0.1% 2-octanone, at most 0.3% 1-heptanol, at most 0.3% octenol (cis/trans), at most 0.1% other alcohols and also at most 0.5% water. The colour number is preferably at maximum APHA 15 and the acid number at maximum 0.2 mg KOH/g.

Suitable polymerization inhibitors, which act as stabilizers, may be for example N-oxides (nitroxyl or N-oxyl radicals, i.e. compounds having at least one NO group) such as 4-hydroxy2,2,6,6-tetramethylpiperidine-N-oxyl (HO-TEMPO), 4-oxo-2,2,6,6-tetramethylpiperidine-N-oxyl, 4-acetoxy-2,2,6,6-tetramethylpiperidine-N-oxyl, 2,2,6,6-tetramethylpiperidine-N-oxyl, bis(1-oxyl2,2,6,6-tetramethylpiperidin-4-yl) sebacate, 4,4′,4″-tris(2,2,6,6-tetramethylpiperidine-N-oxyl) phosphite or 3-oxo-2,2,5,5-tetramethylpyrrolidine-N-oxyl; monohydric or polyhydric phenols optionally having one or more alkyl groups, such as alkylphenols, for example o-, m- or p-cresol (methylphenol), 2-tert-butylphenol, 4-tert-butylphenol, 2,4-di-tert-butylphenol, 2-methyl-4-tertbutylphenol, 2-tert-butyl-4-methylphenol, 2,6-tert-butyl-4-methylphenol, 4-tert-butyl-2,6-dimethylphenol or 6-tert-butyl-2,4-dimethylphenol; quinones such as hydroquinone, hydroquinone monomethyl ether, 2-methylhydroquinone or 2,5-di-tert-butylhydroquinone; hydroxyphenols such as catechol (1,2-dihydroxybenzene) or benzoquinone; aminophenols such as p-aminophenol; nitrosophenols such as p-nitrosophenol; alkoxyphenols such as 2-methoxyphenol (guaiacol, catechol monomethyl ether), 2-ethoxyphenol, 2-isopropoxyphenol, 4-methoxyphenol (hydroquinone monomethyl ether), mono- or di-tert-butyl-4-methoxyphenol; tocopherols such as α-tocopherol and 2,3-dihydro-2,2-dimethyl-7-hydroxybenzofuran (2,2-dimethyl-7-hydroxycumaran), aromatic amines such as N,N-diphenylamine or N-nitrosodiphenylamine; phenylenediamines such as N,N′-dialkyl-p-phenylenediamine, where the alkyl radicals may be the same or different and each independently consist of 1 to 4 carbon atoms and may be straight-chain or branched, for example N,N′-dimethyl-p-phenylenediamine or N,N′-diethyl-p-phenylenediamine, hydroxylamines such as N, N-diethylhydroxylamine, imines such as methylethylimine or methylene violet, sulfonamides such as N-methyl-4-toluenesulfonamide or N-tert-butyl-4-toluenesulfonamide, oximes such as aldoximes, ketoximes or amidoximes such as diethyl ketoxime, methyl ethyl ketoxime or salicyl aldoxime, phosphorus-containing compounds such as triphenylphosphine, triphenyl phosphite, triethyl phosphite, hypophosphorous acid or alkyl esters of phosphorous acids; sulfur-containing compounds such as diphenyl sulfide or phenothiazine; metal salts such as copper or manganese, cerium, nickel, and chromium salts, for example chlorides, sulfates, salicylates, tosylates, acrylates or acetates, for example copper acetate, copper (II) chloride, copper salicylate, cerium (III) acetate or cerium (III) ethylhexanoate, or mixtures thereof.

Preference is given to using as polymerization inhibitor or polymerization inhibitor mixture at least one compound from the group comprising hydroquinone, hydroquinone monomethyl ether, phenothiazine, 4-hydroxy-2,2,6,6-tetramethylpiperidine-N-oxyl, 4-oxo-2,2,6,6-tetramethylpiperidine-N-oxyl, bis(1-oxyl-2,2,6,6-tetramethylpiperidin-4-yl) sebacate, 2-tert-butylphenol, 4-tert-butylphenol, 2,4-di-tert-butylphenol, 2-tert-butyl-4-methylphenol, 6-tert-butyl-2,4-dimethylphenol, 2,6-di-tert-butyl-4-methylphenol, 2-methyl-4-tert-butylphenol, hypophosphorous acid, copper (II) acetate, copper (I) chloride, copper (II) chloride, copper (II) salicylate and cerium (III) acetate.

Particular preference is given to using phenothiazine (PTZ) and/or hydroquinone monomethyl ether (MEHQ) as polymerization inhibitor.

PTZ is especially preferably used as polymerization inhibitor in the case of the esterification or in the case of an optional use of an azeotrope rectification column unit (25). In the case of the optional, downstream process steps, PTZ is also especially preferred used for the cyclohexane separation IV and/or for the 2-octanol separation V.

Very particularly, MEHQ is used as polymerization inhibitor in the optional, downstream process steps in the pure-boiler separation VI and/or high-boiler separation VII.

The polymerization inhibitor is preferably dissolved in one or more liquid organic compounds. The organic compound is preferably 2-octanol and/or 2-octyl (meth)acrylate.

Suitable as esterification catalysts are the common mineral acids and sulfonic acids, preferably sulfuric acid, phosphoric acid, alkylsulfonic acids (e.g. methanesulfonic acid, trifluoromethanesulfonic acid) and arylsulfonic acids (e.g. benzenesulfonic acid, p-toluenesulfonic acid or dodecylbenzenesulfonic acid) or mixtures thereof, but also acidic ion exchangers or zeolites may be used. Particular preference is given to sulfuric acid, methanesulfonic acid, p-toluenesulfonic acid, m-toluenesulfonic acid, o-toluenesulfonic acid or mixtures thereof.

Methanesulfonic acid is especially preferably used as esterification catalyst.

In the process according to the invention, the reactants (meth)acrylic acid and 2-octanol and the azeotroping agent cyclohexane are fed continuously or discontinuously to the one reactor (1) of the reactor unit (24), wherein continuous feeding is preferred.

In the process according to the invention, cyclohexane is used as azeotroping agent. This has a boiling point of 81° C. at standard pressure. The cyclohexane forms a heterogeneous azeotrope with the water of esterification and this heterogeneous azeotrope has a boiling temperature of only 70° C. at standard pressure. With its boiling temperature of 70° C., the heterogeneous azeotrope has a lower boiling point than pure water or than other azeotropes, for example 2-octanol with water of esterification, toluene with water of esterification or octenes with water of esterification. Using cyclohexane, therefore, less energy is required in order to evaporate the heterogeneous azeotrope.

It has been recognized that a lower bottom temperature can be set by feeding an optimal amount of cyclohexane azeotroping agent into the one reactor (1) of the reactor unit (24) than in the case of another azeotroping agent or a non-optimal amount of cyclohexane, such that less secondary components form and at the same time the gas flow, in addition to the gaseous heterogeneous azeotrope, is as pure as possible, and this gas flow thus comprises few other components, such as (meth)acrylic acid for example. Therefore, the bottom temperature required for evaporating the water of esterification may also be influenced significantly by the amount of azeotroping agent used.

A lower bottom temperature reduces, inter alia, the formation of secondary components such as octenes, especially 1-octene and 2-octene. Octenes themselves also act as azeotroping agents, but have a distinctly higher boiling point than 2-octanol. For instance, 1-octene has a boiling temperature of 121° C. at standard pressure. The secondary components formed also increase the separation problem of separating the heterogeneous azeotrope from the reaction mixture and in the pure distillation.

Surprisingly, it has been found that when using the same azeotroping agent concentrations, and other conditions are the same, such as the temperature, reaction time and molar ratios of the feedstocks, the conversion is higher when using cyclohexane, but much fewer secondary components are formed, although the bottom temperature does not differ significantly and the bottom temperature at the end of the process is even highest when using cyclohexane, based on the other azeotroping agents toluene and 2-octanol in excess or no azeotroping agent.

A lot of water remains in the bottoms without azeotroping agent. In order to effectively remove the water, the process would have to be operated under vacuum conditions. However, processes under vacuum are very complex and result in high operating costs and in construction costs of the plant for the process.

The lower boiling temperature of the heterogeneous azeotrope in the one reactor (1) also causes less (meth)acrylic acid to evaporate in the one reactor (1) and/or the reactor heating element (30). This increases the separation performance of separating the heterogeneous azeotrope from the reaction mixture.

In this document, the “reaction mixture” defines a liquid mixture which is formed during the esterification in the one reactor (1) and therefore also comprises the product of value 2-octyl (meth)acrylate.

In this document, the “starting mixture” comprises the reactants 2-octanol and (meth)acrylic acid and in addition the acidic esterification catalyst, the polymerization inhibitor(s) and the azeotroping agent cyclohexane. The starting mixture becomes the reaction mixture due to the esterification.

In this document, the bottom temperature encompasses a temperature range of 90 to 130° C. By changing the bottom temperature within this range, it is also possible to influence the reaction kinetics, the yield/conversion, the selectivity and/or the formation of secondary components etc.

In this document, the term “reactor bottoms” is understood to mean a liquid mixture which is underneath a gas mixture phase in the reactor (1). Therefore, the reactor (1) is only partially filled with a liquid mixture since some of the liquid mixture, preferably the heterogeneous azeotrope, also evaporates during the esterification and thereby fills the reactor space above the liquid mixture.

In this document, the term “rectification column unit” defines one or more rectification columns which may also comprise further standard components such as pressure reducers, flow regulators or sensors. The rectification column unit therefore also encompasses the control thereof. In the case of two or more rectification columns, these may be connected to one another in series or in parallel.

Rectification columns are of a known design per se and comprise the usual apparatus such as an evaporator in the bottoms, an evaporator in the high-boiler outlet or a condenser in the low-boiler outlet, whereby the high-boilers are preferably located in the bottom region and the low-boilers preferably in the overhead region of the rectification column. A portion of the mass flow of the high-boiler outlet is typically fed back to the bottom region of the rectification column. However, it is also possible in principle that the bottom region is heated, for example, via an outer wall heating of the column in the bottom region and/or an evaporator is integrated into the bottom region.

Typically, after condensation in a condenser, the mass flow of the low-boiler outlet is fed back to the top region of the rectification column in the range of 10 to 200% by weight.

The column internals used may in principle be all standard internals, for example trays, structured packings and/or random packings. Among the trays, preference is given to bubble-cap trays, sieve trays, valve trays, Thormann trays and/or dual-flow trays; among the random packings, preference is given to those comprising rings, spirals, saddles or braids.

In this document, components are referred to as low-boilers if the boiling temperature at standard pressure is lower than the boiling temperature of 2-octyl acrylate. Analogously, components are referred to as high-boilers if the boiling temperature at standard pressure is greater than or equal to the boiling temperature of 2-octyl acrylate. The boiling temperature of 2-octyl acrylate is 211° C. at standard pressure.

In this document, the term “reactor having a reactor heating element” generally defines a reactor (1) within a reactor unit (24) having one or more reactor heating element(s) (30) within and/or outside the reactor (1) which heat the reaction mixture. The reactor heating element (30) or one of the reactor heating elements is, for example, an immersion heater in the reactor (1), a tube system comprising coiled tubes or half-coiled tubes arranged on the outer jacket surface of the reactor (1) and/or within the reactor (1), an electrical heating system arranged on the outer jacket surface of the reactor (1) and/or within the reactor (1), an evaporator located outside the reactor (1), wherein the reaction mixture flows at least partially through the evaporator, or a double-walled design of the reactor outer wall, in which a fluid separated from the reaction mixture, such as a liquid, a gas and/or a heating steam, is temperature-controlled and thereby a predetermined heating temperature is set, whereby the reaction mixture is heated in the reactor (1). Two or more reactor heating elements may generally be used to heat the reaction mixture in the reactor (1). For instance, a double-walled design of the reactor outer wall and an evaporator situated outside the reactor (1) may be used to heat the reaction mixture simultaneously or at least partially offset in time.

In this document, the term “evaporator” is also understood to mean a reactor heating element (30). The evaporator may also comprise further standard components such as control valves, pressure reducers, flow regulators or sensors. The evaporator may therefore also comprise a closed-loop control. The term “evaporator” may also generally be understood to mean two or more evaporators which are linked in series or in parallel.

Examples of suitable evaporators are thin-layer, falling film, natural and forced circulation evaporators. The evaporators can be designed as shell-and-tube heat exchangers or plate heat exchangers. Suitable evaporators are known to those skilled in the art and are described, inter alia, in: SPX, Evaporator Handbook, APV, available at https://userpages.umbc.edu/˜dfreyl/ench445/apv_evap.pdf (retrieved on 11.1.2021).

In this document, the term “reactor unit” defines one or more reactors which may also comprise further standard components such as pressure reducers, flow regulators, reactor heating elements, other heating elements, conduits, heat exchangers or evaporators. The reactor unit (24) may also comprise control valves and/or sensors. The reactor unit (24) may thus also comprise process control. It is of course also possible for two or more heat exchangers per reactor or two or more heat exchangers in combination for all reactors of the reactor unit (24) to be present.

In a further preferred configuration, in the case of two or more reactors, the reactors are interconnected with one another in parallel.

In a preferred configuration, the reactor unit (24) comprises two or more reactors in which the reactors are interconnected with one another in series. Thus, a cascade of two or more reactors connected in series is possible, where the cascade means that the discharge flow of one reactor forms the feed flow to the downstream reactor.

In a preferred configuration of the cascade, the components for the esterification are only fed to a first reactor (1) and only the reaction discharge resulting from the esterification is fed in each case to the subsequent reactor.

In a preferred configuration, the one reactor (1) is equipped with internal or external heating coils and/or with a double-walled design of the reactor outer wall.

In a preferred configuration, the one reactor (1) is equipped with an external evaporator, with internal or external heating coils and/or with a double-walled design of the reactor outer wall.

In a preferred configuration, the one reactor (1) is equipped with an external evaporator and with a double-walled design of the reactor outer wall.

In a preferred configuration, the one reactor (1) is equipped with an external evaporator and with internal and/or external heating coils.

In a preferred configuration, the one reactor (1) comprises an external heat exchanger and/or evaporator.

In a preferred embodiment, the one reactor (1) comprises an evaporator. In the case of two or more reactors, each individual reactor comprises its own evaporator.

In a further embodiment, the reactor unit (24) comprises an evaporator, to which all reactors of the reactor unit (24) are connected.

In a preferred embodiment, the one reactor (1) is operated by natural circulation. This has the advantage that the reactor apparatus is cheaper to obtain since, inter alia, it does not require any stirrer or other mechanical aids. The cyclohexane azeotroping agent ensures increased mixing.

In a preferred embodiment, the esterification is operated continuously, in which the reactants and the cyclohexane azeotroping agent are fed continuously and the evaporator unit is also operated continuously.

In a further embodiment, the esterification is operated discontinuously, in which the reactants and the cyclohexane azeotroping agent are fed discontinuously.

In a preferred embodiment of the process, the gaseous heterogeneous azeotrope is fed to an azeotrope rectification column unit (25) downstream of the reactor unit (24), wherein an azeotrope rectification column (4) is located within the azeotrope rectification column unit (25), and the one azeotrope rectification column (4) is operated at an absolute pressure in the range of 0.5 to 2.0 bar and at a bottom temperature in the range of 90 to 130° C., and the heterogeneous azeotrope is removed via the top of the one azeotrope rectification column (4), is condensed in a condenser and is then fed to a phase separator (6), wherein the water of esterification is separated off as lower phase in the phase separator (6), while the cyclohexane is separated off as upper phase in the phase separator (6).

In a further configuration, in the case of two or more reactors, each individual reactor comprises one azeotrope rectification column unit (25) placed thereon.

In a further configuration, in the case of two or more reactors, each individual reactor comprises one azeotrope rectification column unit (25) placed thereon, wherein the azeotrope rectification column unit (25) comprises preferably exactly one azeotrope rectification column (4).

In a particularly preferred configuration, one azeotrope rectification column unit (25) is placed on the entire reactor unit (24), wherein the azeotrope rectification column unit (25) preferably comprises exactly one azeotrope rectification column (4). This has the advantage that only one azeotrope rectification column (4) must be operated for the, or all, reactor(s) and the process is therefore energy- and cost-efficient.

In a preferred embodiment with two or more reactors, the vapors rising from all reactors are fed to a single azeotrope rectification column (4), wherein the liquid discharge of the azeotrope rectification column (4) is only fed back to the first reactor (1). The azeotrope rectification column unit (25) preferably comprises only one azeotrope rectification column.

In a preferred embodiment, the azeotrope rectification column unit (25) is operated continuously.

In a preferred embodiment, the cyclohexane obtained as upper phase in the phase separator (6) is at least partially or fully recycled to the reactor unit (24).

In a preferred configuration, the cyclohexane obtained as upper phase in the phase separator (6) is recycled to the reactor unit (24) to an extent of a proportion by weight in the range of 40 to 100% by weight, preferably in the range of 50 to 99.9% by weight, and the resulting residual proportion of the cyclohexane obtained is fed into the area from below the top to the middle of the one azeotrope rectification column (4).

This has the advantage that the proportion of azeotroping agent in the reactor unit (24) and/or the azeotrope rectification column unit (25) may be adjusted in order to be able to separate off the water of esterification efficiently or also to be able to carry out a specific adjustment of the bottom temperature by feeding cyclohexane to the reactor (1).

In a preferred embodiment, the phase separator (6) is operated continuously by continuously inflowing and outflowing streams.

### Process Stage II: Alkaline Extraction

In a preferred configuration, subsequent to the esterification, an alkaline extraction of the resulting reaction discharge is carried out by means of an alkaline solution, in which, inter alia, the acidic esterification catalyst and unreacted (meth)acrylic acid are neutralized in a neutralization extraction unit (7), and which results in an upper neutralization phase comprising 2-octyl (meth)acrylate, and a lower neutralization phase comprising salts generated by the neutralization and water, and these two phases are therefore present separated from each other. The two neutralization phases are preferably generated by a dispersing apparatus, such as a mixing pump, and are then separated from each other in a phase separator (6) which is located within the neutralization extraction unit (7).

In a preferred embodiment, the neutralization extraction unit (7) is operated continuously by continuously inflowing and outflowing streams.

### Process Stage III: Waterwash Extraction

In a preferred configuration, subsequent to the alkaline extraction of the reaction discharge, the upper neutralization phase obtained from the neutralization extraction unit (7) is extracted with water in a waterwash extraction unit (8), wherein a lower waterwash phase comprising water and salt residues and an upper waterwash phase comprising 2-octyl (meth)acrylate are formed and these two phases are therefore present separated from each other. The two waterwash phases are preferably generated by a dispersing apparatus, such as a mixing pump, and are then separated from each other in a phase separator (6) which is located within the waterwash extraction unit (8).

Both the alkaline extraction (process stage II) and the waterwash extraction (process stage III) may be carried out, for example, in a stirred tank or in another conventional apparatus, for example in a column or in a mixer-settler apparatus. In a mixer-settler or a column, the appropriate processes are preferably carried out continuously. In a stirred tank, the appropriate processes are preferably carried out discontinuously.

There are different dispersion options for the mixer-settler apparatus, such as dispersion in a stirred tank, in a static mixer, in a pipeline or in a mixing pump and a phase separator. Mixer and settler can be constructed more or less independently of each other. The mixing process can be adjusted, for example, by the correct choice of stirrer, stirring speed, power input and/or throughput. In the case of a mixing pump, the power input can be preferably adjusted.

Both the alkaline extraction (process stage II) and the waterwash extraction (process stage III) can preferably be an extraction column, a centrifugal extractor or a mixer-settler apparatus.

In a particularly preferred embodiment, in the case of an alkaline extraction (process stage II), the resulting reaction discharge is dispersed by a pump, for example a mixing pump, with a dilute alkali and then fed to a phase separator (6), which separates the upper and lower neutralization phases from each other.

The process technology used can be all extraction and wash methods and extraction apparatuses known per se, for example those that have been described in Ullmann's Encyclopedia of Industrial Chemistry, 6th edition, 1999 Electronic Release, Chapter: Liquid—Liquid Extraction—Apparatus. For example, these can be single or multi-stage extractions, preferably single-stage extractions, which may be operated in co-current or counter-current mode.

Waterwash is then preferably used if salts are to be separated off.

In a preferred embodiment, the waterwash extraction unit (8) is operated continuously by continuously inflowing and outflowing streams.

In a particularly preferred embodiment, a static mixer is used which disperses the upper neutralization phase obtained from the neutralization extraction unit (7) with separately supplied water. Subsequently, the mixture dispersed by the static mixer is fed to a phase separator (6), in which a lower waterwash phase comprising water and salt residues and an upper waterwash phase comprising 2-octyl (meth)acrylate are formed.

The following process stages IV to VII are preferably operated continuously.

### Process Stage IV: Cyclohexane Removal

In a preferred configuration, subsequent to the alkaline extraction of the reaction discharge, the cyclohexane azeotroping agent is separated off from the upper neutralization phase in an azeotrope rectification column unit, wherein an azeotrope rectification column (9) is located within the azeotrope rectification column unit, and the one azeotrope rectification column (9) is operated at a reduced absolute pressure in the range of 0.05 to 0.9 bar and at a bottom temperature in the range of 70 to 120° C., and an organic phase is withdrawn via the top of the one azeotrope rectification column (9) comprising the azeotroping agent cyclohexane, octenes, 2-octanol and a mass flow proportion of 2-octyl (meth)acrylate in the range of 0.1 to 5.0%, based on the upper neutralization phase fed in, wherein the components withdrawn via the head form an azeotrope mass flow, wherein this is condensed and is fed to the one reactor (1) of the reactor unit (24) in the range of 50 to 100%, wherein the residual azeotrope mass flow proportion is condensed and is fed to the top of the azeotrope rectification column (9), whereas high-boilers comprising di(meth)acrylic acid esters and oxy esters, such as the alkoxyalkyl esters of (meth)acrylic acid, and also 2-octanol and 2-octyl (meth)acrylate are removed through a bottom discharge of the one azeotrope rectification column (9), wherein in the range of 20 to 95% of the mass flow of the bottom discharge flows through an evaporator and is subsequently recycled to the one azeotrope rectification column (9).

In a preferred configuration, subsequent to the waterwash extraction, the cyclohexane azeotroping agent is separated off from the upper waterwash phase in an azeotrope rectification column unit, wherein an azeotrope rectification column (9) is located within the azeotrope rectification column unit, and the one azeotrope rectification column (9) is operated at a reduced absolute pressure in the range of 0.05 to 0.9 bar and at a bottom temperature in the range of 70 to 120° C., and an organic phase is withdrawn via the top of the one azeotrope rectification column (9) comprising the azeotroping agent cyclohexane, octenes, 2-octanol and a mass flow proportion of 2-octyl (meth)acrylate in the range of 0.1 to 5.0%, based on the upper waterwash phase fed in, wherein the components withdrawn via the head form an azeotrope mass flow, wherein this is condensed and is fed to the one reactor (1) of the reactor unit (24) in the range of 50 to 100% by weight, wherein the residual azeotrope mass flow proportion is condensed and is fed back to the top of the azeotrope rectification column (9), whereas high-boilers comprising di(meth)acrylic acid esters and oxy esters, such as the alkoxyalkyl esters of (meth)acrylic acid, and also 2-octanol and 2-octyl (meth)acrylate are removed through a bottom discharge of the one azeotrope rectification column (9), wherein in the range of 20 to 95% of the mass flow of the bottom discharge flows through an evaporator and is subsequently recycled to the one azeotrope rectification column (9).

In a particularly preferred configuration, exactly one azeotrope rectification column (9) is located within the azeotrope rectification column unit.

### Process Stage V: 2-Octanol Separation

In a particularly preferred configuration, subsequent to the separation of the cyclohexane azeotroping agent, the 2-octanol is separated off from the mass flow produced by the bottom discharge of the one azeotrope rectification column (9) in a 2-octanol rectification column unit, wherein a 2-octanol rectification column (10) is located within the 2-octanol rectification column unit, and the one
2-octanol rectification column (10) is operated at a reduced absolute pressure in the range of 0.005 to 0.10 bar and at a bottom temperature in the range of 70 to 130° C., and the mass flow produced by the bottom discharge of the one azeotrope rectification column (9) is metered in in the area below the top to the middle of the 2-octanol rectification column (10), and the components discharged via the top of the one 2-octanol rectification column (10) are condensed and are fed to the one reactor (1) of the reactor unit (24) in the range of 20 to 50%, based on the mass flow of the components discharged via the top, wherein the discharged components consist of 2-octanol in the range of 2 to 40% by weight. On the other hand, 2-octyl (meth)acrylate and high-boilers comprising di(meth)acrylic acid esters and oxy esters are withdrawn through a bottom discharge of the 2-octanol rectification column (10), wherein 20 to 95% of the mass flow of the bottom discharge flows through an evaporator and is subsequently fed back to the one 2-octanol rectification column (10).

In a preferred configuration, subsequent to the separation of the cyclohexane azeotroping agent, the 2-octanol is separated from the mass flow produced by the bottom discharge of the one azeotrope rectification column (9) in a 2-octanol evaporator unit, wherein a 2-octanol evaporator (110) is located within the 2-octanol evaporator unit, and the one 2-octanol evaporator (110) is operated at a reduced absolute pressure in the range of 0.005 to 0.10 bar and at a bottom temperature in the range of 70 to 130° C., and the mass flow produced by the bottom discharge of the one azeotrope rectification column (9) is metered into a 2-octanol inlet of the one 2-octanol evaporator, and the components discharged via a 2-octanol evaporator (110) outlet of the one 2-octanol evaporator are fed to the reactor unit (24) in the range of 20 to 50%, based on the mass flow of the components discharged at the 2-octanol evaporator outlet, wherein these discharged components consist of 2-octanol in the range of 2 to 40% by weight. On the other hand, 2-octyl (meth)acrylate and high-boilers comprising di(meth)acrylic acid esters and oxy esters are withdrawn through a bottom discharge of the one 2-octanol evaporator, wherein in the range of 20 to 95% of the mass flow of the bottom discharge flows through an evaporator and is subsequently fed back to the one 2-octanol evaporator (110).

In a preferred configuration, after the cyclohexane separation, the 2-octanol and at the same time the 2-octyl (meth)acrylate are separated from the residual components comprising cyclohexane in a dividing wall column (131).

In a preferred configuration, the dividing wall column (131) is provided with a vertical dividing wall (138) which prevents cross-mixing of liquid and vapor streams in partial areas. The dividing wall (138), which may consist of a flat metal sheet, divides the column longitudinally in its central region into a feeding section (140) and a withdrawal section (141).

In a preferred configuration, the mixture to be separated, comprising low-boilers, 2-octyl (meth)acrylate and high-boilers, is fed to the feeding section (140) and 2-octyl (meth)acrylate is withdrawn from the withdrawal section (141) in vapor or liquid form. The low-boilers are separated off via the top of the dividing wall column (131) and the high-boilers via the bottom of the dividing wall column (131).

The energy requirement and investment costs are ca. 25% lower than for a conventional column arrangement having two rectification columns.

### Process Stage VI: Pure Boiler Separation (Separation of the Target Product 2-Octyl (Meth)Acrylate)

In a preferred configuration, subsequent to the separation of the 2-octanol, a pure boiler separation is carried out from the mass flow produced by the bottom discharge of the one 2-octanol rectification column (10) in a pure boiler rectification column unit, wherein a pure boiler rectification column (11) is located within the pure boiler rectification column unit, and the one pure boiler rectification column (11) is operated at a reduced absolute pressure in the range of 0.002 to 0.05 bar and at a bottom temperature in the range of 80 to 130° C., and the mass flow produced by the bottom discharge of the one 2-octanol rectification column (10) is metered in the region below the top to the middle of the one pure boiler rectification column (11), and to stabilize the one pure boiler rectification column (11), 2-octyl (meth)acrylate and a polymerization inhibitor each in the range of 0.01 to 1.0%, based on the mass flow produced by the bottom discharge of the one 2-octanol rectification column (10), are metered in to the region from the bottom up to the middle of the one pure boiler rectification column (11), and the 2-octyl (meth)acrylate is withdrawn via the top of the one pure boiler rectification column (11), while high-boilers comprising di(meth)acrylic acid esters and oxy esters, such as the alkoxyalkyl esters of (meth)acrylic acid, are withdrawn through a bottom discharge of the one pure boiler rectification column (11).

In a particularly preferred configuration, subsequent to the separation of the 2-octanol, a pure boiler separation is carried out from the mass flow produced by the bottom discharge of the one 2-octanol evaporator in a pure boiler evaporator unit, wherein a pure boiler evaporator is located within the pure boiler evaporator unit, and the one pure boiler evaporator (111) is operated at a reduced absolute pressure in the range of 0.002 to 0.05 bar and at a bottom temperature in the range of 80 to 130° C., and the mass flow produced by the bottom discharge of the one 2-octanol rectification column (10) or of the one 2-octanol evaporator is metered into a pure boiler evaporator inlet of the one pure boiler evaporator unit, and to stabilize the one pure boiler evaporator, 2-octyl (meth)acrylate and a polymerization inhibitor each in the range of 0.01 to 1.0%, based on the mass flow produced by the bottom discharge of the one 2-octanol evaporator, are metered into a pure boiler evaporator inlet of the one pure boiler evaporator, and the 2-octyl (meth)acrylate is withdrawn via a pure boiler evaporator outlet of the one pure boiler evaporator, while high-boilers comprising di(meth)acrylic acid esters and oxy esters, such as the alkoxyalkyl esters of (meth)acrylic acid, are withdrawn through a bottom discharge of the one pure boiler evaporator.

In a very particularly preferred configuration, the pure boiler evaporator unit comprises exactly one pure boiler evaporator.

### Process Step VII: High-Boiler Separation (Di(Meth)Acrylic Acid Esters and Oxy Esters Such as the Alkoxyalkyl Esters of (Meth)Acrylic Acid)

In a preferred configuration, subsequent to the pure boiler separation, high-boilers are separated off from the mass flow produced by the bottom discharge of the one pure boiler rectification column (11) or the one pure boiler evaporator, in a high-boiler rectification column unit, wherein a high-boiler rectification column (12) is located within the high-boiler rectification column unit, and the one high-boiler rectification column (12) is operated at a reduced absolute pressure in the range of 0.002 to 0.05 bar and at a bottom temperature in the range of 80 to 150° C., and the mass flow produced by the bottom discharge of the one pure boiler rectification column (11) is metered into the region from the bottom up to the middle of the one high-boiler rectification column (12), and the takeoff withdrawn via the top of the one high-boiler rectification column (12) is condensed and is fed into the region from the bottom up to the middle of the one pure boiler rectification column (11) or to the pure boiler evaporator inlet of the one pure boiler evaporator.

In a particularly preferred configuration, subsequent to the pure boiler separation, high-boilers are separated off from the mass flow produced by the bottom discharge of the one pure boiler rectification column (11) or the one pure boiler evaporator, in a high-boiler evaporator unit, wherein a high-boiler evaporator is located within the high-boiler evaporator unit, and the one high-boiler evaporator is operated at a reduced absolute pressure in the range of 0.002 to 0.05 bar and at a bottom temperature in the range of 80 to 150° C., and the mass flow produced by the bottom discharge of the one pure boiler evaporator is metered into a high-boiler evaporator inlet of the one high-boiler evaporator, and the takeoff withdrawn from the one high-boiler evaporator is condensed and is fed back into the region from the bottom up to the middle of the one pure boiler rectification column (11) or to the pure boiler evaporator inlet of the one pure boiler evaporator.

In a very particularly preferred configuration, the high-boiler evaporator unit comprises exactly one high-boiler evaporator.

### Further Preferred Embodiments of Process Stages I and II

In a preferred configuration, the bottom temperature in the one reactor (1) of the reactor unit (24) is in the range of 100 to 125° C., preferably 110 to 115° C.

In a preferred configuration, the esterification is carried out at an absolute pressure in the range of 0.8 to 1.5 bar, particularly preferably 0.9 to 1.2 bar.

In a preferred configuration, less than 1.0% octenes are formed in the bottoms of the reactor (1), based on the sum total of the mass flows of 2-octanol (13), acidic esterification catalyst (15), polymerization inhibitors (31) and (meth)acrylic acid (14) fed to the reactor (1). This may be achieved, inter alia, when applying the configurations from the two preceding paragraphs.

In a preferred configuration, cyclohexane is fed to the one reactor (1) of the reactor unit (24) in an amount in the range of 100 to 600% by weight, preferably in the range of 200 to 500% by weight, particularly preferably in the range of 350 to 450% by weight, based in each case on the sum total of the amounts by mass of 2-octanol (13) and (meth)acrylic acid (14) fed to the one reactor (1) of the reactor unit (24).

In a preferred configuration, cyclohexane at a concentration in the range of 10 to 90% by weight is additionally metered into the one reactor (1) of the reactor unit (24), wherein the percentage by weight of the concentration refers to the components present in the one reactor (1) of the reactor unit (24) including the cyclohexane metered in.

In a preferred configuration, the proportion of catalyst in the one reactor (1) of the reactor unit (24) is at maximum 10% by weight, based on the sum of the components of 2-octanol and (meth)acrylic acid present in the reactor (1) of the reactor unit (24).

In a preferred configuration, the alkaline solution in process stage (II) is aqueous NaOH, which comprises NaOH in the range of 5 to 25% by weight, preferably NaOH in the range of 10 to 20% by weight.

In a preferred configuration, the reactor contents are circulated in the one reactor (1) by natural circulation or forced circulation.

In a preferred configuration, the total components flowing into the one reactor (1) of the reactor unit (24) without the cyclohexane azeotroping agent have the following proportions by weight:

| | |
|---|---|
| 2-octanol: | 40.00 to 84.39% by weight |
| (meth)acrylic acid: | 15.00 to 59.39% by weight |
| acidic esterification catalyst: | 0.50 to 10.00% by weight |
| polymerization inhibitor: | 0.01 to 1.00% by weight |
| residual components: | 0.10 to 5.00% by weight. |

The azeotroping agent cyclohexane is added in an amount such that a concentration of cyclohexane forms in the reactor (1) of the reactor unit (24) in the range of 10 to 90% by weight, wherein these figures of the proportion by weight relate to the components present in the reactor (1) of the reactor unit (24) including the cyclohexane.

In addition, in this preferred configuration without the azeotroping agent cyclohexane and without the water of esterification, the total components flowing out of the reactor (1) of the reactor unit (24), namely the resulting liquid reaction discharge plus the portion evaporated from the reaction mixture and discharged from the reactor unit (24), have the following proportions by weight:

| | |
|---|---|
| 2-octyl (meth)acrylate: | 50.00 to 95.00% by weight |
| 2-octanol: | 1.00 to 30.00% by weight |
| (meth)acrylic acid: | 1.00 to 15.00% by weight |
| acidic esterification catalyst: | 0.50 to 10.00% by weight |
| polymerization inhibitor: | 0.01 to 1.00% by weight |
| residual components: | 2.49 to 10.00% by weight. |

The heterogeneous azeotrope formed by the cyclohexane and the water of esterification flows out of the reactor (1) of the reactor unit (24) at a concentration in the range of 10 to 50% by weight, where these figures of the percentage by weight refer to the total components flowing out including the cyclohexane and the water of esterification.

In a particularly preferred configuration, the total components flowing into the one reactor (1) of the reactor unit (24) without the cyclohexane azeotroping agent have the following proportions by weight:

| | |
|---|---|
| 2-octanol: | 50.00 to 74.39% by weight |
| (meth)acrylic acid: | 25.00 to 45.00% by weight |
| acidic esterification catalyst: | 0.50 to 5.00% by weight |
| polymerization inhibitor: | 0.01 to 1.00% by weight |
| residual components: | 0.10 to 2.00% by weight. |

The azeotroping agent cyclohexane is added in an amount such that a concentration of cyclohexane forms in the reactor (1) of the reactor unit (24) in the range of 10 to 50% by weight, wherein these figures of the proportion by weight relate to the components present in the reactor (1) of the reactor unit (24) including the cyclohexane.

In addition, in this particularly preferred configuration, without the azeotroping agent cyclohexane and without the water of esterification, the total components flowing out of the one reactor (1) of the reactor unit (24), namely the resulting liquid reaction discharge plus the portion evaporated from the reaction mixture and discharged from the reactor unit (24), have the following proportions by weight:

| | |
|---|---|
| 2-octyl (meth)acrylate: | 70.00 to 95.00% by weight |
| 2-octanol: | 1.00 to 15.00% by weight |
| (meth)acrylic acid: | 1.00 to 10.00% by weight |
| acidic esterification catalyst: | 0.50 to 10.00% by weight |
| polymerization inhibitor: | 0.01 to 1.00% by weight |
| residual components: | 2.49 to 10.00% by weight. |

The heterogeneous azeotrope formed by the cyclohexane and the water of esterification flows out of the one reactor (1) of the reactor unit (24) at a concentration in the range of 10 to 50% by weight, where these figures of the percentage by weight refer to the total components flowing out including the cyclohexane and the water of esterification.

The invention will be discussed in more detail below with reference to the drawings. The drawings are to be understood as diagrammatic illustrations. They do not constitute a limitation of the invention, for example with regard to specific dimensions or design versions.

### SHOWN ARE

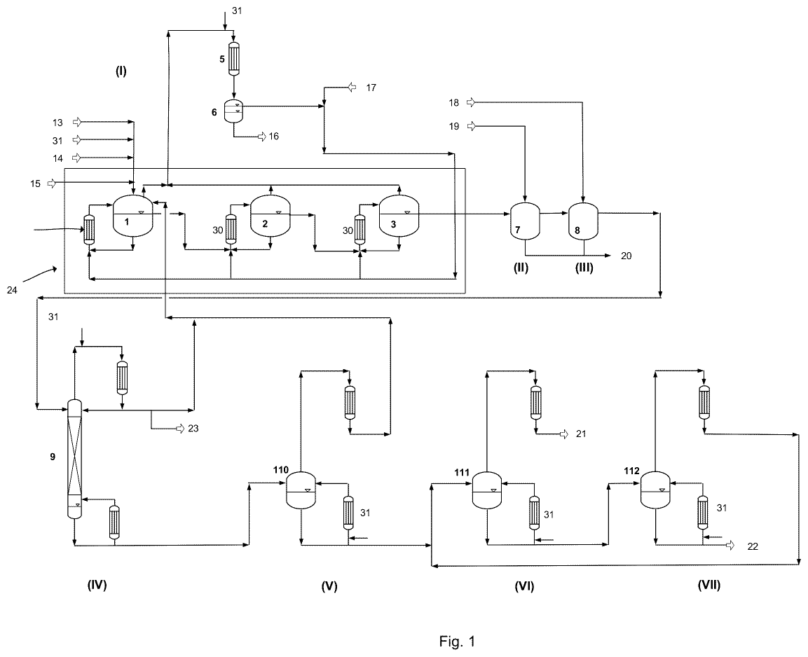

FIG. 1: Process outline for producing 2-octyl (meth)acrylate based on an evaporator as reactor heating element 30 for separating the heterogeneous azeotrope.

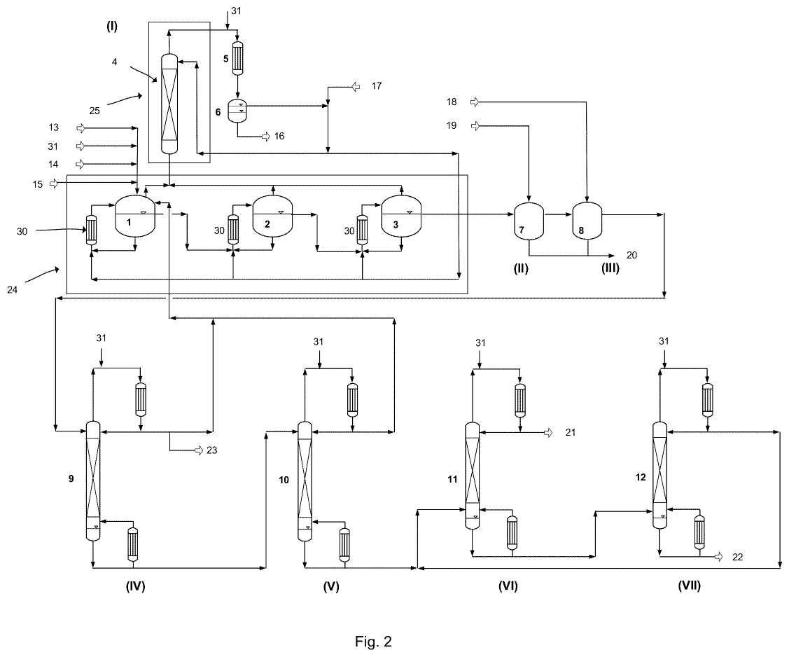

FIG. 2: Process outline for producing 2-octyl (meth)acrylate based on an evaporator as reactor heating element 30 and an azeotrope rectification column unit 25 for separating the heterogeneous azeotrope.

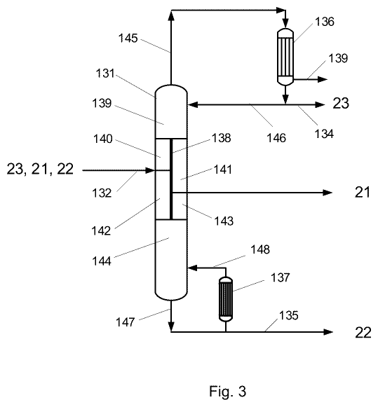

FIG. 3: Process outline for the dividing wall column.

### LIST OF THE REFERENCE NUMBERS USED

1 Reactor 1
2 Reactor 2
3 Reactor 3
4 Azeotrope rectification column
5 Condenser for the heterogeneous azeotrope
6 Phase separator for the heterogeneous azeotrope
7 Neutralization extraction unit
8 Waterwash extraction unit
9 Azeotrope rectification column
10 2-Octanol rectification column
110 2-Octanol evaporator
11 Pure boiler rectification column
111 Pure boiler evaporator
12 High-boiler rectification column
112 High-boiler evaporator
13 2-Octanol feed
14 (Meth)acrylic acid feed
15 Acidic esterification catalyst feed
16 Water discharge, which is discharged from the phase separator (6).
17 Cyclohexane feed
18 Water feed for the waterwash
19 Alkaline solution feed
20 Discharge of water with salt residues from the alkaline and/or waterwash extraction
21 Discharge of the 2-octyl (meth)acrylate product of value
22 Discharge of high-boilers which form in the high-boiler removal in the bottoms.
23 Discharge of low-boilers, which are removed from the process in the cyclohexane
25 separation after discharge of the cyclohexane-enriched mass flow.
24 Reactor unit
25 Azeotrope rectification column unit
30 Reactor heating element
31 Feed of polymerization inhibitor and/or further stabilizers
131 Dividing wall column
132 Mixture to be separated in the dividing wall column
134 Distillate stream of the dividing wall column
135 Substream of the dividing wall column
136 Condenser of the dividing wall column
137 Evaporator of the dividing wall column
138 Dividing wall of the dividing wall column
139 Common upper column region of the dividing wall column
140 Feeding section of the dividing wall column
141 Withdrawal section of the dividing wall column
142 Stripping section of the dividing wall column
143 Rectifying section of the dividing wall column
144 Lower column region of the dividing wall column
145 Vapor stream of the dividing wall column
146 Return stream of the dividing wall column
147 Liquid stream of the dividing wall column

FIG. 1 shows a process outline for producing 2-octyl (meth)acrylate with the respective process 50 stages, wherein each reactor comprises an evaporator as reactor heating element for separating off the heterogeneous azeotrope.

### Process Stage (I): Esterification

The esterification is reacted in a reactor unit 24 composed of three cascaded reactors 1, 2, 3. The reactant streams flow into the first reactor 1 of the cascade: 2-octanol 13, (meth)acrylic acid 14, acidic esterification catalyst 15, polymerization inhibitors 31 and the azeotroping agent cyclohexane 17. After the first reactor 1 of the cascade reaches a predetermined level, a mass flow flows to the second reactor 2 of the cascade. After the second reactor 2 of the cascade reaches a predetermined level, a mass flow flows to the third reactor 3 of the cascade. After the third reactor 3 reaches a predetermined level, a resulting reaction discharge flows out of the reaction unit. All three reactors 1, 2, 3 of the reactor unit 24 each comprise their own evaporator 30, through which the reaction mixture flows, is heated and is then fed back to the respective reactor. In the evaporator 30, the water of esterification formed during esterification and the azeotroping agent cyclohexane partially evaporate as a heterogeneous azeotrope (cyclohexane/water of esterification).

A gas stream enriched with the heterogeneous azeotrope is withdrawn from the individual reactors 1, 2, 3 and cooled until condensation occurs by a downstream condenser 5. In a downstream phase separator 6, an organic upper phase and an aqueous lower phase is formed. The aqueous lower phase is discarded from the process. The organic upper phase comprises up to 50 to 99.9% by weight cyclohexane. This organic upper phase is fed to the first reactor 1 of the cascade and to the azeotrope rectification column 4 in the region from the top to the middle of the column.

### Process Stage (II): Alkaline Extraction

The resulting reaction discharge from the esterification stage is neutralized with an alkaline solution 19 in a neutralization extraction unit 7, wherein the acidic esterification catalyst and the unreacted (meth)acrylic acid are neutralized. As a result, an upper neutralization phase comprising 2-octyl (meth)acrylate, and a lower neutralization phase comprising salts generated by the neutralization and water, is formed. The lower neutralization phase 20 is discarded from the process.

### Process Stage (III): Waterwash Extraction

Following the alkaline extraction, the upper neutralization phase is fed to a waterwash extraction unit 8, in which, with the addition of water 18, a lower waterwash phase 20 comprising water and salt residues and an upper waterwash phase comprising 2-octyl (meth)acrylate are formed. The lower waterwash phase 20 is discarded from the process.

### Process Stage (IV): Cyclohexane Removal

The upper waterwash phase from the waterwash extraction is fed to an azeotrope rectification column 9. Low-boilers and cyclohexane are removed via the top of this column 9.

The components removed via the top of the column 9 are condensed in the range of 95 to 99.9% by weight and are fed back to the top region of the azeotrope rectification column 9. The remaining residual portion, after separation of gaseous low-boilers 23 such as octenes, is fed back to the first reactor 1 of the cascade. Cyclohexane is typically conveyed from the column 9 to the first reactor 1 of the cascade in the range of 2 to 20% by weight, based on the total mass of cyclohexane fed overall to the process.

### Process Stage (V): 2-Octanol Separation

The proportion of the bottom discharge that is not fed back to the azeotrope rectification column 9 is supplied to a 2-octanol evaporator 110. 2-Octanol, in an amount in the range of 20 to 60% by weight based on the bottom discharge of the azeotrope rectification column 9 fed to the 2-octanol evaporator 110, is withdrawn via the 2-octanol evaporator 110, condensed and fed entirely to the first reactor 1 of the cascade.

### Process Stage (VI): Pure Boiler Separation

The portion of the bottom discharge that is not fed back to the 2-octanol evaporator 110 is fed to a pure boiler evaporator 111. The 2-octyl (meth)acrylate product of value is withdrawn via a vapor stream of the pure boiler evaporator 111 in the range of 80 to 95% by weight, based on the bottom discharge fed to the pure boiler evaporator 111, is condensed and entirely fed back to the pure boiler evaporator 111. The remaining residual portion of the bottom discharge of the octanol evaporator 110 is condensed and fed entirely to a high-boiler evaporator 112.

### Process Stage (VII): High-Boiler Separation

The corresponding residual portion of the bottom discharge of the pure boiler evaporator 111 is fed to a high-boiler evaporator 112. Low-boilers are withdrawn via a vapor stream of the high-boiler evaporator 112 in the range of 40 to 90% by weight, based on the bottom discharge of the pure boiler evaporator 111 fed to the high-boiler evaporator 112, and entirely fed back to the pure boiler evaporator 111. From 1 to 10% by weight, based on the circulation of the high-boiler evaporator 112, of the high-boilers 22 present in the bottoms of the high-boiler evaporator 112 are discharged from the process, the resulting remaining portion of the high-boilers 22 being evaporated and fed back to the high-boiler evaporator 112.

FIG. 2 shows a process outline for producing 2-octyl (meth)acrylate with the respective process stages, wherein an azeotrope rectification column 4 for separating off the heterogeneous azeotrope is used.

### Process Stage (I): Esterification

The esterification is reacted in a reactor unit 24 composed of three cascaded reactors 1, 2, 3. The reactant streams flow into the first reactor 1 of the cascade: 2-octanol 13, (meth)acrylic acid 14, acidic esterification catalyst 15, polymerization inhibitors 31 and the azeotroping agent cyclohexane 17. After the first reactor 1 of the cascade reaches a predetermined level, a mass flow flows to the second reactor 2 of the cascade. After the second reactor 2 of the cascade reaches a predetermined level, a mass flow flows to the third reactor 3 of the cascade. After the third reactor 3 reaches a predetermined level, a resulting reaction discharge flows out of the reaction unit. All three reactors 1, 2, 3 of the reactor unit 24 each comprise their own evaporator 30, through which the reaction mixture flows, is heated and is then fed back to the respective reactor. In the evaporator 30, the water of esterification formed during esterification and the azeotroping agent cyclohexane at least partially evaporate as a heterogeneous azeotrope (cyclohexane/water of esterification).

A gas stream enriched with the heterogeneous azeotrope is withdrawn from the individual reactors 1, 2, 3 and fed to a downstream azeotrope rectification column 4. The azeotrope rectification column 4 is placed on the reactor unit 24. Polymerization inhibitors 31 are fed to the top of the azeotrope rectification column 4.

The gas stream enriched with heterogeneous azeotrope is fed via the top of the azeotrope rectification column 4 and is cooled until condensation occurs by a condenser 5. In a downstream phase separator 6, an organic upper phase and an aqueous lower phase is formed.

The aqueous lower phase is discarded from the process. The organic upper phase comprises up to 50 to 99.9% by weight cyclohexane. This organic upper phase is fed to the first reactor 1 of the cascade and to the azeotrope rectification column 4 in the region from the top to the middle of the column.

### Process Stage (II): Alkaline Extraction

The resulting reaction discharge from the esterification stage is neutralized with an alkaline solution in a neutralization extraction unit 7, wherein the acidic esterification catalyst and unreacted (meth)acrylic acid are neutralized. As a result, an upper neutralization phase comprising 2-octyl (meth)acrylate, and a lower neutralization phase comprising salts generated by the neutralization and water, is formed. The lower neutralization phase is discarded from the process.

### Process Stage (III): Waterwash Extraction

Following the alkaline extraction, the upper neutralization phase is fed to a waterwash extraction unit 8, in which, with the addition of water 18, a lower waterwash phase 20 comprising water and salt residues and an upper waterwash phase comprising 2-octyl (meth)acrylate are formed. The lower waterwash phase 20 is discarded from the process.

### Process Stage (IV): Cyclohexane Removal

The upper waterwash phase from the waterwash extraction is fed to an azeotrope rectification column 9. Low-boilers and cyclohexane are removed via the top of this column 9.

The components removed via the top of column 9 are condensed in the range of 95 to 99.9% by weight and are fed back to the top region of the azeotrope rectification column 9. The resulting residual portion, after separation of low-boilers 23 such as octenes, is fed to the first reactor 1 of the cascade. Cyclohexane is typically conveyed from column 9 to the first reactor 1 of the cascade in the range of 2 to 20% by weight, based on the total mass of cyclohexane fed overall to the process.

### Process Stage (V): 2-Octanol Separation

The proportion of the bottom discharge that is not fed back to the azeotrope rectification column 9 is fed to a 2-octanol rectification column 10. 2-Octanol is withdrawn via the top of this column in an amount in the range of 20 to 60% by weight, based on the bottom discharge of the azeotrope rectification column 9 fed to the column 10. The components withdrawn via the top of column 10 are fully condensed and fed back to the 2-octanol rectification column 10 in the top 10 region in the range of 0 to 50% by weight, while the resulting remaining portion of the top takeoff is fed to the first reactor 1 of the cascade.

### Process Stage (VI): Pure Boiler Separation

The portion of the bottom discharge that is not fed back to the 2-octanol rectification column 10 is fed to a pure boiler rectification column 11. The 2-octyl (meth)acrylate product of value is withdrawn via the top of this pure boiler rectification column 11 in the range of 80 to 95% by weight, based on the bottom discharge fed to the column 11. The discharge via the top of the pure boiler rectification column 11 is fully condensed and fed back to the top region of the pure boiler rectification column 11 in the range of 0 to 60% by weight. The resulting residual portion of the bottom discharge of the octanol rectification column 10 is fed to a high-boiler separation.

### Process (Step VII): High-Boiler Separation

The corresponding residual portion of the bottom discharge of the pure boiler rectification column 11 is fed to a high-boiler rectification column 12. Low-boilers in the range of 40 to 300% by weight, based on the bottom discharge fed to the high-boiler rectification column 12, are withdrawn via the top of this high-boiler rectification column 12 and a portion of the takeoff in the range of 40 to 90% by weight is fed back to the pure boiler rectification column 11, the resulting residual portion being fed back to the high-boiler rectification column 12 in the top region of the column 12. The high-boilers 22 present in the bottoms of the high-boiler rectification column 12 are at least partially discharged from the process, wherein the resulting residual portion of the high-boilers 22 is evaporated and fed back to the bottom region of the high-boiler rectification column 12.

FIG. 3 shows a process outline of the dividing wall column.

The process outline shows a dividing wall column 131 with dividing wall 138, which divides the dividing wall column 131 into a common upper column region 139, a feeding section 140, 142, with rectifying section 140 and stripping section 142, a withdrawal section 141, 143 with a stripping section 141 and a rectifying section 143 and also a common lower column region 144. The mixture to be separated 132, comprising the low-boilers, 2-octyl (meth)acrylate and high-boilers, enters the dividing wall column 131 between the column sections 140 and 142. The pure product 133 is withdrawn between the column sections 141 and 143, preferably in liquid form. The vapor stream 145 obtained at the column head is partially condensed in the condenser 136, which may be supplemented by an aftercooler, and divided into the return stream 146 and the distillate stream 134. The uncondensed fraction from the condenser 136 comprises the low-boiling impurities and is taken off in vapor form as stream 139. The liquid 147 at the lower column end is partially evaporated in an evaporator 137 and is fed back to the column via the conduit 148. A substream 135 comprising the high-boiling impurities is discharged. The evaporator 137 may be designed as a natural circulation evaporator or as a forced circulation evaporator, in the latter case a circulation pump additionally being required for the liquid stream 147. Particularly advantageous with regard to avoiding undesirable polymerization reactions is to use a falling film evaporator or thin-layer evaporator instead of the forced circulation evaporator, since with this design the shortest residence times are possible.

To reduce the residence time of the liquid in the evaporator system, it is favorable to arrange the level control not in the lower column cap but in the feed conduit of the liquid stream 147.

### EXAMPLES

Both in the following examples and throughout this document, the term “qualitative GC” refers to gas chromatography (GC) in which the individual components have been assigned by comparison of retention times. The concentration of the respective components in the mixture is assumed to be the peak percentage area (area %).

Both in the following examples and throughout this document, the term “quantitative GC” refers to gas chromatography (GC) with quantification by reference to the internal standard n-tetradecane. The components 2-octyl acrylate, cyclohexane and 2-octanol were quantified. The concentration of the respective components in the mixture is obtained as percentage by weight (% by weight).

The expected amount of water corresponds to the amount of water at full conversion or infinite reaction time and results from the sum of the water contents of the starting materials used and the water of esterification to be formed at 100% conversion. The sum total of these water contents is also referred to in this document as water of reaction. The maximum water of esterification to be formed may be calculated via the moles of water formed which are equivalent to the moles of acrylic acid used. A slight carry-over of acrylic acid into the aqueous phase was not taken into account in the balance sheet.

### Example 1: Discontinuous Esterification with Cyclohexane (Process Stage I)

A 0.75 L heatable double-walled reactor equipped with thermal sensor, anchor stirrer, water separator, jacketed coil condenser and air inlet was initially charged with 300 g of 2-octanol, 0.6 g of phenothiazine, 0.6 g of a 1% by weight solution of HO-TEMPO in 2-octyl acrylate, 130 g of cyclohexane, 0.015 g of CuCl and 0.240 g of 50% hypophosphorous acid. 161 g of acrylic acid were then metered in, where the acrylic acid was stabilized with 200 ppm MeHQ. 6.4 g of 100% methanesulfonic acid were added. The total batch size was 599 g.

The reaction mixture was heated at a bath temperature of 125° C. The reaction time started on heating. In the course of the reaction, bottom samples were taken and analyzed by gas chromatography in order to monitor the course of the reaction.

After 0.58 h, at a bottom temperature of 99° C., water began to pass over and the distillation time started. The reaction was terminated after a distillation time of 6 h, equivalent to 6.59 h reaction time, when a bottom temperature of 119° C. had been reached. 35.6 g of water had been distilled off, corresponding to 88.2% of the expected amount of water.

The reaction mixture was analyzed by gas chromatography after a distillation time of 6 h. 2-Octanol and 2-octyl acrylate were quantified. After a distillation time of 6 h, equivalent to 6.59 h reaction time, the mixture comprised 71.54% by weight octyl acrylate and 5.75% by weight 2-octanol. The reaction mixture comprised 0.27 area % octenes, 1.78 area % diacrylic acid esters and 1.85 area % oxyesters.

The conversion was 92.56% based on 2-octanol.

### Comparative Example 1: Discontinuous Esterification with Toluene (Process Stage I)

A 0.75 L heatable double-walled reactor equipped with thermal sensor, anchor stirrer, water separator, jacketed coil condenser and air inlet was initially charged with 300 g of 2-octanol, 0.6 g of phenothiazine, 0.6 g of a 1% by weight solution of HO-TEMPO in 2-octyl acrylate, 130 g of toluene, 0.015 g of CuCl and 0.240 g of 50% hypophosphorous acid. 161 g of acrylic acid were then metered in, where the acrylic acid was stabilized with 200 ppm MeHQ. 6.4 g of 100% methanesulfonic acid were added. The total batch size was 599 g.

The reaction mixture was heated at a bath temperature of 125° C. The reaction time started on heating. In the course of the reaction, bottom samples were taken and analyzed by gas chromatography in order to monitor the course of the reaction.

After 0.92 h, at a bottom temperature of 108° C., water began to pass over and the distillation time started. The reaction was terminated after a distillation time of 6 h, equivalent to 6.92 h reaction time, when a bottom temperature of 116° C. had been reached. 17.7 g of water had been distilled off, corresponding to 43.9% of the expected amount of water.

The reaction mixture was analyzed by gas chromatography after a distillation time of 6 h. 2-Octanol and 2-octyl acrylate were quantified. After a distillation time of 6 h, equivalent to 6.92 h reaction time, the mixture comprised 58.38% by weight octyl acrylate and 13.35% by weight 2-octanol. The reaction mixture comprised 0.15 area % octenes, 2.18 area % diacrylic acid esters and 1.57 area % oxyesters.

The conversion was 81.39% based on 2-octanol.

### Comparative Example 2: Discontinuous Esterification with 2-Octanol as Azeotroping Agent (Process Stage I)

A 0.75 L heatable double-walled reactor equipped with thermal sensor, anchor stirrer, water separator, jacketed coil condenser and air inlet was initially charged with 300 g of 2-octanol, 0.6 g of phenothiazine, 0.6 g of a 1% by weight solution of HO-TEMPO in 2-octyl acrylate, a further 130 g of 2-octanol, 0.015 g of CuCl and 0.240 g of 50% hypophosphorous acid. 161 g of acrylic acid were then metered in, where the acrylic acid was stabilized with 200 ppm MeHQ. 6.4 g of 100% methanesulfonic acid were added. The total batch size was 599 g.

The reaction mixture was heated at a bath temperature of 125° C. The reaction time started on heating. In the course of the reaction, bottom samples were taken and analyzed by gas chromatography in order to monitor the course of the reaction.

After 1.22 h, at a bottom temperature of 112° C., water began to pass over and the distillation time started. The reaction was terminated after a distillation time of 6 h, equivalent to 7.22 h reaction time, when a bottom temperature of 114° C. had been reached. 12.8 g of water had been distilled off, corresponding to 31.6% of the expected amount of water.

The reaction mixture was analyzed by gas chromatography after a distillation time of 6 h. 2-Octanol and 2-octyl acrylate were quantified. After a distillation time of 6 h, equivalent to 7.22 h reaction time, the mixture comprised 54.64% by weight octyl acrylate and 33.01% by weight 2-octanol. The reaction mixture comprised 0.09 area % octenes, 1.11 area % diacrylic acid esters and 2.20 area % oxyesters.

The conversion was 62.35% based on 2-octanol.

### Comparative Example 3: Discontinuous Esterification without Azeotroping Agent (Process Stage I)

A 0.75 L heatable double-walled reactor equipped with thermal sensor, anchor stirrer, water separator, jacketed coil condenser and air inlet was initially charged with 383 g of 2-octanol, 0.6 g of phenothiazine, 0.6 g of a 1% solution of hydroxy-tempo in 2-octyl acrylate, 0.015 g of CuCl and 0.240 g of 50% hypophosphorous acid. 206 g of acrylic acid were then metered in, where the acrylic acid was stabilized with 200 ppm MeHQ. 8.2 g of 100% methanesulfonic acid were added. The total batch size was 599 g.

The reaction mixture was heated at a bath temperature of 125° C. The reaction time started on heating. In the course of the reaction, bottom samples were taken and analyzed by gas chromatography in order to monitor the course of the reaction.

After 1.17 h, at a bottom temperature of 111° C., water began to pass over and the distillation time started. The reaction was terminated after a distillation time of 6 h, equivalent to 7.17 h reaction time, when a bottom temperature of 113° C. had been reached. 11.0 g of water had been distilled off, corresponding to 21.4% of the expected amount of water.

The reaction mixture was analyzed by gas chromatography after a distillation time of 6 h. 2-Octanol and 2-octyl acrylate were quantified. After a distillation time of 6 h, equivalent to 7.17 h reaction time, the mixture comprised 59.16% by weight octyl acrylate and 21.33% by weight 2-octanol. The reaction mixture comprised 0.06 area % octenes, 2.57 area % diacrylic acid esters and 2.00 area % oxyesters.

The conversion was 73.5% based on 2-octanol.

### Abstract

All experiments were conducted at a bath temperature of 125° C. Acid and alcohol were used in the same molar ratio. An exception here is the experiment with 2-octanol as azeotroping agent, in which the molar ratio of acrylic acid to 2-octanol was 1:1.45. The catalyst concentration based on the acid used was always the same.

Azeotroping agents in the experiments were cyclohexane, toluene and 2-octanol, which was present in addition to the 2-octanol for the reaction. In addition, in one experiment, no azeotroping agent was used.

### Tabular Summary of the Results

Table 1 shows an overview of the reaction in the case of Example 1, where the azeotroping agent cyclohexane was used. Listed are the distillation time, the reaction time, the bottom temperature, the water of reaction and the conversion. The water-based conversion was calculated as follows:

$$water - based conversion [ \% ] = \frac{water of reaction [ g ] ( t = x h )}{water of reaction [ g ] ( t = ∞ )} \times 100.$$

Here, water of reaction where t=∞ signifies the maximum amount of water of reaction to be expected which is present in the case of complete conversion in the esterification. The water of reaction where t=xh corresponds to the amount of water of reaction present at time point xhours due to the esterification and the water content of the starting materials used.

TABLE 1
Overview of the reaction in the case of Example 1, where the azeotroping agent
cyclohexane was used. Listed are the distillation time, the reaction time,
the bottom temperature, the water of reaction and the water-based conversion.

| | Distillation time [h] | | | | | | |
|---|---|---|---|---|---|---|---|
| | 0 | 1 | 2 | 3 | 4 | 5 | 6 |
| Reaction time [h] | 0.58 | 1.58 | 2.58 | 3.58 | 4.58 | 5.58 | 6.58 |
| Bottom temperature [° C.] | 99.2 | 107.4 | 112.8 | 116.1 | 117.7 | 118.5 | 118.9 |
| Water of reaction [g] | | 16.57 | 28.63 | 33.08 | 34.44 | 35.12 | 35.6 |
| Water-based conversion [%] | | 41.07 | 70.95 | 81.98 | 85.35 | 87.04 | 88.23 |

Table 2 shows an overview of the GC values of the qualitative GC measurements at various distillation times for Example 1, where cyclohexane was used as azeotroping agent. Listed are the sum of 1-octene and 2-octene, the diacrylic acid esters (DIAA esters), the oxyesters, the 2-octyl acrylate content, the conversion, calculated from the values of the qualitative GC, and the ratio of 2-octyl acrylate to the sum total of the by-products 1-octene, 2-octene, DIAA esters and oxyesters.

The conversion was calculated as follows:

$$conversion [ \% ] = \frac{GC - Value ( 2 - octyl acrylate ) [ area \% ]}{\begin{matrix}GC - Value ( 2 - octyl acrylate ) [ area \% ] + \\ GC - Value ( 2 - Octanol ) [ area \% ]\end{matrix}} \times 100.$$

Here, the GC value (2-octyl acrylate) [area %] refers to the area percentage of 2-octyl acrylate measured by qualitative GC. By analogy, the GC value (2-octanol) [area %] refers to the area percentage of 2-octanol measured by qualitative GC.

TABLE 2
Overview of the GC values of the qualitative GC measurements at various distillation times for Example 1, where
cyclohexane was used as azeotroping agent. Listed are the sum of 1-octene and 2-octene, the DIAA esters, the
oxyesters, the 2-octyl acrylate content, the conversion calculated from the values of the qualitative GC and
the ratio of 2-octyl acrylate to the sum total of the by-products 1-octene, 2-octene, DIAA esters and oxyesters.

| | Distillation time [h] | | | | | |
|---|---|---|---|---|---|---|
| | 1 | 2 | 3 | 4 | 5 | 6 |
| Sum total 1-octene, 2-octene [area %] | 0 | 0 | 0.1 | 0.15 | 0.21 | 0.27 |
| DIAA esters [area %] | 1.05 | 1.34 | 1.55 | 1.7 | 1.76 | 1.78 |
| Oxyesters [area %] | 0.64 | 1.06 | 1.39 | 1.65 | 1.76 | 1.85 |
| 2-Octyl acrylate content [area %] | 40.14 | 55.61 | 64.07 | 70.88 | 70.06 | 69.89 |
| Conversion [%] | 57.07 | 77.35 | 86.36 | 90.13 | 91.59 | 92.36 |
| Ratio of 2-octyl acrylate to the sum total | 100:4.2 | 100:4.3 | 100:4.7 | 100:4.9 | 100:5.3 | 100:5.6 |
| of by-products 1-octene, 2-octene, DIAA | | | | | | |
| esters, oxyesters DIAA esters, oxyesters | | | | | | |

Table 3 shows an overview of various distillation times in the case of Example 1, where the azeotroping agent cyclohexane was used. Listed are the fraction by weight of 2-octyl acrylate, the fraction by weight of 2-octanol and the molar conversion. Here, the molar conversion is calculated from the values of the quantitative GC.

The molar conversion was calculated as follows:

$$Molar conversion [ \% ] = \frac{\begin{matrix}GC - Value ( 2 - octyl acrylate ) [ \% of weight ] / \\ W\_Mol ( 2 - octyl acrylate ) [ \frac{g}{mol} ]\end{matrix}}{\begin{matrix}\begin{matrix}\begin{matrix}GC - Value ( 2 - octyl acrylate ) [ \% by weight ] / \\ W\_Mol ( 2 - octyl acrylate ) [ \frac{g}{mol} ]\end{matrix} + \\ GC - Value ( 2 - Octanol ) [ \% by weight ] /\end{matrix} \\ W\_mol ( 2 - Octanol ) [ \frac{g}{mol} ]\end{matrix}} \times 100 ,$$

Using the abbreviation W_Mol as molecular weight.

Here, the GC value (2-octyl acrylate) [% by weight] refers to the proportion of 2-octyl acrylate quantified by gas chromatography. By analogy, the GC value (2-octanol) [% by weight] refers to the proportion of 2-octanol quantified by gas chromatography.

TABLE 3
Overview of the GC values at various distillation times for Example
1, where cyclohexane was used as azeotroping agent. Listed are
the fraction by weight of 2-octyl acrylate, the fraction by weight
of 2-octanol and the molar conversion. Here, the molar conversion
is calculated from the values of the quantitative GC.

| Distillation time [h] | 3 | 4 | 5 | 6 |
|---|---|---|---|---|
| 2-Octyl acrylate [% by weight] | 66.61 | 70.27 | 70.70 | 71.54 |
| 2-Octanol [% by weight] | 10.20 | 7.66 | 6.38 | 5.75 |
| Molar conversion [%] | 82.18 | 86.63 | 88.67 | 89.79 |

Table 4 shows an overview of the reaction in the case of comparative example 1, where the azeotroping agent toluene was used. Listed are the distillation time, the reaction time, the bottom temperature, the water of reaction and the conversion. The water-based conversion was calculated as follows:

$$water - based conversion [ \% ] = \frac{water of reaction [ g ] ( t = x h )}{water of reaction [ g ] ( t = ∞ )} \times 100.$$

Here, water of reaction where t=∞ signifies the maximum amount of water of reaction to be expected which is present in the case of complete conversion in the esterification. The water of reaction where t=xh corresponds to the amount of water of reaction present at time point xhours due to the esterification and the water content of the starting materials used.

TABLE 4
Overview of the reaction in the case of comparative example 1, where the azeotroping
agent toluene was used. Listed are the distillation time, the reaction time, the
bottom temperature, the water of reaction and the water-based conversion.

| | Distillation time [h] | | | | | | |
|---|---|---|---|---|---|---|---|
| | 0 | 1 | 2 | 3.00 | 4 | 5 | 6 |
| Reaction time [h] | 0.92 | 1.92 | 2.92 | 3.92 | 4.92 | 5.92 | 6.92 |
| Bottom temperature [° C.] | 107.8 | 111.4 | 113.1 | 114.1 | 115.1 | 115.6 | 115.9 |
| Water of reaction [g] | | 5.97 | 10.95 | 13.87 | 16.2 | 17.09 | 17.73 |
| Water-based conversion [%] | | 14.80 | 27.14 | 34.37 | 40.15 | 42.35 | 43.94 |

Table 5 shows an overview of the GC values of the qualitative GC measurements at various distillation times for comparative example 1, where toluene was used as azeotroping agent. Listed are the sum of 1-octene and 2-octene, the DIAA esters, the oxyesters, the 2-octyl acrylate content, the conversion calculated from the values of the qualitative GC and the ratio of 2-octyl acrylate to the sum total of the by-products 1-octene, 2-octene, DIAA esters and oxyesters.

The conversion was calculated as follows:

$$conversion [ \% ] = \frac{GC - Value ( 2 - octyl acrylate ) [ area \% ]}{\begin{matrix}GC - Value ( 2 - octyl acrylate ) [ area \% ] + \\ GC - Value ( 2 - Octanol ) [ area \% ]\end{matrix}} \times 100.$$

Here, the GC value (2-octyl acrylate) [area %] refers to the area percentage of 2-octyl acrylate measured by qualitative GC. By analogy, the GC value (2-octanol) [area %] refers to the area percentage of 2-octanol measured by qualitative GC.

TABLE 5
Overview of the GC values of the qualitative GC measurements at various distillation times
for comparative example 1, where toluene was used as azeotroping agent. Listed are the sum
of 1-octene and 2-octene, the DIAA esters, the oxyesters, the 2-octyl acrylate content, the
conversion calculated from the values of the qualitative GC and the ratio of 2-octyl acrylate
to the sum total of the by-products 1-octene, 2-octene, DIAA esters and oxyesters.

| Distillation time [h] | | | | | |
|---|---|---|---|---|---|
| 1 | 2 | 3 | 4.00 | 5 | 6 |

| | | | | |
|---|---|---|---|---|
| Sum total 1-octene, 2-octene [area %] | 0.08 | 0.11 | 0.12 | 0.15 |
| DIAA esters [area %] | 1.59 | 1.85 | 2.00 | 2.18 |
| Oxyesters [area %] | 1.24 | 1.38 | 1.47 | 1.57 |
| 2-Octyl acrylate content [area %] | 51.68 | 54.43 | 55.84 | 56.78 |
| Conversion [%] | 73.96 | 77.56 | 79.28 | 80.79 |
| Ratio of 2-octyl acrylate to the sum total | 100:5.6 | 100:6.1 | 100:6.4 | 100:6.9 |
| of by-products 1-octene, 2-octene, DIAA | | | | |
| esters, oxyesters DIAA esters, oxyesters | | | | |

Table 6 shows an overview at various distillation times in the case of comparative example 1, where the azeotroping agent toluene was used. Listed are the fraction by weight of 2-octyl acrylate, the fraction by weight of 2-octanol and the molar conversion. Here, the molar conversion is calculated from the values of the quantitative GC.

The molar conversion was calculated as follows:

$$Molar conversion [ \% ] = \frac{\begin{matrix}GC - Value ( 2 - octyl acrylate ) [ \% of weight ] / \\ W\_Mol ( 2 - octyl acrylate ) [ \frac{g}{mol} ]\end{matrix}}{\begin{matrix}\begin{matrix}\begin{matrix}GC - Value ( 2 - octyl acrylate ) [ \% by weight ] / \\ W\_Mol ( 2 - octyl acrylate ) [ \frac{g}{mol} ]\end{matrix} + \\ GC - Value ( 2 - Octanol ) [ \% by weight ] /\end{matrix} \\ W\_mol ( 2 - Octanol ) [ \frac{g}{mol} ]\end{matrix}} \times 100 ,$$

Using the abbreviation W_Mol as molecular weight.

Here, the GC value (2-octyl acrylate) [% by weight] refers to the proportion of 2-octyl acrylate quantified by gas chromatography. By analogy, the GC value (2-octanol) [% by weight] refers to the proportion of 2-octanol quantified by gas chromatography.

TABLE 6
Overview of the GC values at various distillation times for comparative
example 1, where toluene was used as azeotroping agent. Listed
are the fraction by weight of 2-octyl acrylate, the fraction by
weight of 2-octanol and the molar conversion. Here, the molar conversion
is calculated from the values of the quantitative GC.

| Distillation time [h] | 3 | 4 | 5 | 6 |
|---|---|---|---|---|
| 2-Octyl acrylate [% by weight] | 52.87 | 55.90 | 57.62 | 58.38 |
| 2-Octanol [% by weight] | 17.87 | 15.53 | 14.46 | 13.35 |
| Molar conversion [%] | 67.64 | 71.77 | 73.79 | 75.55 |

Table 7 shows an overview of the reaction in the case of comparative example 2, where the azeotroping agent 2-octanol was used. Listed are the distillation time, the reaction time, the bottom temperature, the water of reaction and the conversion. The water-based conversion was calculated as follows:

$$water - based conversion [ \% ] = \frac{water of reaction [ g ] ( t = x h )}{water of reaction [ g ] ( t = ∞ )} \times 100.$$

Here, water of reaction where t=∞ signifies the maximum amount of water of reaction to be expected which is present in the case of complete conversion in the esterification. The water of reaction where t=xh corresponds to the amount of water of reaction present at time point xhours due to the esterification and the water content of the starting materials used.

TABLE 7
Overview of the reaction in the case of comparative example 2, where the azeotroping
agent 2-octanol was used. Listed are the distillation time, the reaction time,
the bottom temperature, the water of reaction and the water-based conversion.

| | Distillation time [h] | | | | | | |
|---|---|---|---|---|---|---|---|
| | 0 | 1 | 2 | 3 | 4 | 5 | 6 |
| Reaction time [h] | 1.22 | 2.22 | 3.22 | 4.22 | 5.22 | 6.22 | 7.22 |
| Bottom temperature [° C.] | 112.1 | 111.1 | 111.6 | 112.1 | 113.2 | 113.7 | 114.3 |
| Water of reaction [g] | | 1.24 | 3.34 | 6.09 | 8.96 | 11.02 | 12.76 |
| Water-based conversion [%] | | 3.07 | 8.28 | 15.09 | 22.21 | 27.31 | 31.62 |

Table 8 shows an overview of the GC values of the qualitative GC measurements at various distillation times for comparative example 2, where 2-octanol was used as azeotroping agent. Listed are the sum of 1-octene and 2-octene, the DIAA esters, the oxyesters, the 2-octyl acrylate content, the conversion calculated from the values of the qualitative GC and the ratio of 2-octyl acrylate to the sum total of the by-products 1-octene, 2-octene, DIAA esters and oxyesters. The conversion was calculated as follows:

$$conversion [ \% ] = \frac{GC - Value ( 2 - octyl acrylate ) [ area \% ]}{\begin{matrix}GC - Value ( 2 - octyl acrylate ) [ area \% ] + \\ GC - Value ( 2 - Octanol ) [ area \% ]\end{matrix}} \times 100.$$

Here, the GC value (2-octyl acrylate) [area %] refers to the area percentage of 2-octyl acrylate measured by qualitative GC. By analogy, the GC value (2-octanol) [area %] refers to the area percentage of 2-octanol measured by qualitative GC.

TABLE 8
Overview of the GC values of the qualitative GC measurements at various distillation times
for comparative example 2, where 2-octanol was used as azeotroping agent. Listed are the
sum of 1-octene and 2-octene, the DIAA esters, the oxyesters, the 2-octyl acrylate content,
the conversion calculated from the values of the qualitative GC and the ratio of 2-octyl
acrylate to the sum total of the by-products 1-octene, 2-octene, DIAA esters and oxyesters.

| Distillation time [h] | | | | | |
|---|---|---|---|---|---|
| 1 | 2 | 3 | 4 | 5 | 6 |

| | | | | |
|---|---|---|---|---|
| Sum total 1-octene, 2-octene [area %] | 0.12 | 0.08 | 0.04 | 0.09 |
| DIAA esters [area %] | 0.92 | 0.99 | 1.04 | 1.11 |
| Oxyesters [area %] | 1.62 | 1.82 | 2.00 | 2.20 |
| 2-Octyl acrylate content [area %] | 51.46 | 53.71 | 55.20 | 56.46 |
| Conversion [%] | 55.40 | 57.82 | 59.57 | 60.99 |
| Ratio of 2-octyl acrylate to the sum total | 100:5.2 | 100:5.4 | 100:5.6 | 100:6.0 |
| of by-products 1-octene, 2-octene, DIAA | | | | |
| esters, oxyesters DIAA esters, oxyesters | | | | |

Table 9 shows an overview at various distillation times in the case of comparative example 2, where the azeotroping agent 2-octanol was used. Listed are the fraction by weight of 2-octyl acrylate, the fraction by weight of 2-octanol and the molar conversion. Here, the molar conversion is calculated from the values of the quantitative GC.

The molar conversion was calculated as follows:

$$Molar conversion [ \% ] = \frac{\begin{matrix}GC - Value ( 2 - octyl acrylate ) [ \% of weight ] / \\ W\_Mol ( 2 - octyl acrylate ) [ \frac{g}{mol} ]\end{matrix}}{\begin{matrix}\begin{matrix}\begin{matrix}GC - Value ( 2 - octyl acrylate ) [ \% by weight ] / \\ W\_Mol ( 2 - octyl acrylate ) [ \frac{g}{mol} ]\end{matrix} + \\ GC - Value ( 2 - Octanol ) [ \% by weight ] /\end{matrix} \\ W\_mol ( 2 - Octanol ) [ \frac{g}{mol} ]\end{matrix}} \times 100 ,$$

Using the abbreviation W_Mol as molecular weight.

Here, the GC value (2-octyl acrylate) [% by weight] refers to the proportion of 2-octyl acrylate quantified by gas chromatography. By analogy, the GC value (2-octanol) [% by weight] refers to the proportion of 2-octanol quantified by gas chromatography.

TABLE 9
Overview of the GC values at various distillation times for comparative
example 2, where 2-octanol was used as azeotroping agent. Listed
are the fraction by weight of 2-octyl acrylate, the fraction by
weight of 2-octanol and the molar conversion. Here, the molar conversion
is calculated from the values of the quantitative GC.

| Distillation time [h] | 3 | 4 | 5 | 6 |
|---|---|---|---|---|
| 2-Octyl acrylate [% by weight] | 49.31 | 51.63 | 53.43 | 54.66 |
| 2-Octanol [% by weight] | 37.50 | 35.49 | 34.17 | 33.01 |
| Molar conversion [%] | 56.81 | 50.69 | 52.48 | 53.91 |

Table 10 shows an overview of the reaction in the case of comparative example 3, where no azeotroping agent was used. Listed are the distillation time, the reaction time, the bottom temperature, the water of reaction and the conversion. The water-based conversion was calculated as follows:

$$water - based conversion [ \% ] = \frac{water of reaction [ g ] ( t = x h )}{water of reaction [ g ] ( t = ∞ )} \times 100.$$

Here, water of reaction where t=∞ signifies the maximum amount of water of reaction to be expected which is present in the case of complete conversion in the esterification. The water of reaction where t=xh corresponds to the amount of water of reaction present at time point xhours due to the esterification and the water content of the starting materials used.

TABLE 10
Overview of the reaction in the case of comparative example 3, where no azeotroping
agent was used. Listed are the distillation time, the reaction time, the bottom
temperature, the water of reaction and the water-based conversion.

| | Distillation time [h] | | | | | | |
|---|---|---|---|---|---|---|---|
| | 0 | 1 | 2 | 3 | 4 | 5 | 6 |
| Reaction time [h] | 1.17 | 2.17 | 3.17 | 4.17 | 5.17 | 6.17 | 7.17 |
| Bottom temperature [° C.] | 110.7 | 110.2 | 110.6 | 111.1 | 111.7 | 112.0 | 112.5 |
| Water of reaction [g] | | 1.33 | 3.23 | 5.67 | 8.21 | 9.51 | 11.00 |
| Water-based conversion [%] | | 2.58 | 6.27 | 11.01 | 15.95 | 18.47 | 21.37 |

Table 11 shows an overview of the GC values of the qualitative GC measurements at various distillation times for comparative example 3, where no azeotroping agent was used. Listed are the sum of 1-octene and 2-octene, the DIAA esters, the oxyesters, the 2-octyl acrylate content, the conversion calculated from the values of the qualitative GC and the ratio of 2-octyl acrylate to the sum total of the by-products 1-octene, 2-octene, DIAA esters and oxyesters.

The conversion was calculated as follows:

$$conversion [ \% ] = \frac{GC - Value ( 2 - octyl acrylate ) [ area \% ]}{\begin{matrix}GC - Value ( 2 - octyl acrylate ) [ area \% ] + \\ GC - Value ( 2 - Octanol ) [ area \% ]\end{matrix}} \times 100.$$

Here, the GC value (2-octyl acrylate) [area %] refers to the area percentage of 2-octyl acrylate measured by qualitative GC. By analogy, the GC value (2-octanol) [area %] refers to the area percentage of 2-octanol measured by qualitative GC.

TABLE 11
Overview of the GC values of the qualitative GC measurements at various distillation times
for comparative example 11, where no azeotroping agent was used. Listed are the sum of
1-octene and 2-octene, the DIAA esters, the oxyesters, the 2-octyl acrylate content, the
conversion calculated from the values of the qualitative GC and the ratio of 2-octyl acrylate
to the sum total of the by-products 1-octene, 2-octene, DIAA esters and oxyesters.

| Distillation time [h] | | | | | |
|---|---|---|---|---|---|
| 1 | 2 | 3 | 4 | 5 | 6 |

| | | | | |
|---|---|---|---|---|
| Sum total 1-octene, 2-octene [area %] | 0.06 | 0.04 | 0.07 | 0.06 |
| DIAA esters [area %] | 1.82 | 2.13 | 2.34 | 2.57 |
| Oxyesters [area %] | 1.60 | 1.78 | 1.88 | 2.00 |
| 2-Octyl acrylate content [area %] | 59.31 | 61.47 | 62.91 | 63.58 |
| Conversion [%] | 67.13 | 69.69 | 71.2 | 72.41 |
| Ratio of 2-octyl acrylate to the sum total | 100:5.9 | 100:6.4 | 100:6.8 | 100:7.3 |
| of by-products 1-octene, 2-octene, DIAA | | | | |
| esters, oxyesters DIAA esters, oxyesters | | | | |

Table 12 shows an overview at various distillation times in the case of comparative example 3, where no azeotroping agent was used. Listed are the fraction by weight of 2-octyl acrylate, the fraction by weight of 2-octanol and the molar conversion. Here, the molar conversion is calculated from the values of the quantitative GC.

The molar conversion was calculated as follows:

$$Molar conversion [ \% ] = \frac{\begin{matrix}GC - Value ( 2 - octyl acrylate ) [ \% of weight ] / \\ W\_Mol ( 2 - octyl acrylate ) [ \frac{g}{mol} ]\end{matrix}}{\begin{matrix}\begin{matrix}\begin{matrix}GC - Value ( 2 - octyl acrylate ) [ \% by weight ] / \\ W\_Mol ( 2 - octyl acrylate ) [ \frac{g}{mol} ]\end{matrix} + \\ GC - Value ( 2 - Octanol ) [ \% by weight ] /\end{matrix} \\ W\_mol ( 2 - Octanol ) [ \frac{g}{mol} ]\end{matrix}} \times 100 ,$$

Using the abbreviation W_Mol as molecular weight.

Here, the GC value (2-octyl acrylate) [% by weight] refers to the proportion of 2-octyl acrylate quantified by gas chromatography. By analogy, the GC value (2-octanol) [% by weight] refers to the proportion of 2-octanol quantified by gas chromatography.

TABLE 12
Overview of the GC values at various distillation times for comparative
example 3, where no azeotroping agent was used. Listed are the
fraction by weight of 2-octyl acrylate, the fraction by weight
of 2-octanol and the molar conversion. Here, the molar conversion
is calculated from the values of the quantitative GC.

| Distillation time [h] | 3 | 4 | 5 | 6 |
|---|---|---|---|---|
| 2-Octyl acrylate [% by weight] | 54.47 | 56.46 | 57.96 | 59.16 |
| 2-Octanol [% by weight] | 25.28 | 23.19 | 22.32 | 21.33 |
| Molar conversion [%] | 60.35 | 63.24 | 64.73 | 66.21 |

The concentration of by-products increases as the reaction progresses. At similar conversion, the formation of the by-products is significantly lower when using cyclohexane as azeotroping agent compared to using toluene and 2-octanol as azeotroping agents or in the case that no azeotroping agent is used.

Table 13 below compares the ratio of 2-octyl acrylate to the sum of the by-products 1-octene, 2-octene, DIAA ester and oxyester at similar conversions, each determined from the GC values of the qualitative GC. In this case, the examples are shown with the respective azeotroping agents cyclohexane, toluene and 2-octanol and also the example without azeotroping agent.

TABLE 13
Comparison of the ratio of 2-octyl acrylate to the sum of the
by-products 1-octene, 2-octene, DIAA ester and oxyester at
similar conversions. In this case, the examples are shown with
the respective azeotroping agents cyclohexane, toluene and
2-octanol and also the example without azeotroping agent.

| | | | | |
|---|---|---|---|---|
| Cyclohexane | Conversion [%] | 57.07 | 77.35 | 86.36 |
| | Ratio of 2-octyl acrylate to | 100:4.2 | 100:4.3 | 100:4.7 |
| | the sum of by-products | | | |
| | 1-octene, 2-octene, | | | |
| | DIAA ester, oxyester | | | |
| Toluene | Conversion [%] | | 77.56 | 80.79 |
| | Ratio of 2-octyl acrylate to | | 100:6.1 | 100:6.9 |
| | the sum of by-products | | | |
| | 1-octene, 2-octene, | | | |
| | DIAA ester, oxyester | | | |
| 2-Octanol | Conversion [%] | 55.40 | | |
| | Ratio of 2-octyl acrylate to | 100:5.2 | | |
| | the sum of by-products | | | |
| | 1-octene, 2-octene, | | | |
| | DIAA ester, oxyester | | | |
| Without | Conversion [%] | | 72.41 | |
| azeotroping | | | | |
| agent | | | | |
| | Ratio of 2-octyl acrylate to | | 100:7.3 | |
| | the sum of by-products | | | |
| | 1-octene, 2-octene, | | | |
| | DIAA ester, oxyester | | | |

This results in the following advantages when using cyclohexane when comparing the results with comparative examples 1, 2 and 3:
Using cyclohexane as azeotroping agent, the highest conversion is generated with the same energy input. This results in fewer losses of (meth)acrylic acid in the subsequent alkaline extraction unit (process stage II). In addition, less 2-octanol is recycled, and smaller reactors with the same throughput.
using cyclohexane as azeotroping agent, a more rapid start to the reflux is achieved after starting heating. As a result, a shorter reaction time and less energy is required.
the cyclohexane azeotroping agent separates off the water most efficiently.
using cyclohexane as azeotroping agent, the 2-octyl acrylate content in the reaction mixture is greatest for the same reaction volume and same distillation time. Therefore, the absolute space-time yield is the highest.
using cyclohexane as azeotroping agent, fewer by-products are formed at the same conversion.

### Comparative Example 4: Esterification, Alkaline Extraction and Waterwash without Azeotroping Agent (Process Stage I, II and III)

A heatable 0.75 L double-walled reactor equipped with, inter alia, a thermal sensor, an anchor stirrer, a glass attachment from Normag (source: www.normag-glas.de) comprising a jacketed coil condenser, a drainage spout having two needle valves, a vacuum frame having two spindle valves as shut-off valve and a venting valve, and a vacuum pump and air inlet, was initially charged with 300 g of 2-octanol, 0.52 g of phenothiazine, 0.012 g of CuCl and 0.207 g of 50% hypophosporous acid. 209 g of acrylic acid were then metered in, where the acrylic acid was stabilized with 200 ppm MeHQ. 6.4 g of 100% methanesulfonic acid were added. This was heated at a bath temperature of 130° C., whereby the reaction time was started. After one hour, at a bottom temperature of 112° C., water began to pass over and the distillation time started. After a distillation time of 2 h, reduction of the absolute pressure was started in 50 mbar steps down to 100 mbar. The internal temperature of the bottoms was at most 122° C. After a distillation time of 4.1 h, the reaction was terminated. 71.8 g had been distilled off. After cooling, the reaction mixture was extracted with water, with 12.5% NaOH solution and then again with water. After phase separation, 50 mg of MeHQ was added to the organic phase. This gave 403 g of 2-octyl acrylate at a purity of 85.4 GC area %. 3.5 area % 2-octanol was still present and also 5.9 area % diacrylic acid ester and 3.1 area % oxyester. Ethanol was added to the biphasic distillate to form a single phase and analyzed by qualitative GC and also acid-base titration and Karl-Fischer titration. The composition of the distillate before addition of ethanol was determined mathematically based on these results. The distillate was composed of 51.8% water, 29.5% acrylic acid and also 6.3% 2-octanol and 9.5% 2-octyl acrylate, also 2.2% octenes.

### Example 2: Esterification, Alkaline Extraction and Waterwash with Cyclohexane (Process Stage I, II and III)

A heatable 4 L double-walled reactor equipped, inter alia, with a thermal sensor, a disk stirrer, a water separator, a jacketed coil condenser, an air inlet, was initially charged with 1503 g of 2-octanol, 4.01 g of phenothiazine and 1.94 g of MeHQ. 1082 g of acrylic acid were then metered in, where the acrylic acid was stabilized with 200 ppm MeHQ. 65.7 g of p-toluenesulfonic acid monohydrate and 1189 g of cyclohexane were added and the mixture was heated. Water passed over at a bottom temperature in the range of 92 to 104° C. After 7.2 h, the reaction was stopped. After cooling, the reaction mixture was extracted with water, with 12.5% NaOH solution and then again with water. After phase separation, 250 mg of MeHQ were added to the organic phase and concentrated in vacuo. This gave 2072 g of 2-octyl acrylate at a purity of 93.2 GC area %. 2.6 area % 2-octanol was still present and also 2.0 area % diacrylic acid ester and 1.3 area % oxyester. GC analysis of the organic liquid present in the water separator showed 99.38 GC area % cyclohexane and also 0.47 area % acrylic acid, 0.03 area % 2-octanol, 0.1 area % 2-octyl acrylate and 0.01 area % octenes. Karl-Fischer titration of the aqueous distillate gave 85% by weight water.

### Example 3: Esterification, Alkaline Extraction and Waterwash with Cyclohexane (Process Stage I, II and III)

In this example 3, a reactor 1 and an azeotrope rectification column 4, inter alia, was used. Process stage I, II and Ill for esterification, for alkaline extraction and for waterwash extraction were operated continuously.

A heatable 1.6 L double-walled glass reactor 1 was equipped, inter alia, with a heatable cover and also a 3-stage crossbeam stirrer.

The azeotrope rectification column 4 used was a double-walled, mirrored glass column with dimensions 50 cm (height)×43 mm (diameter) with a structured packing from Montz (source: www.montz.de/gewebepackung-typ-3a, accessed on Nov. 11, 2021) (Montz Pack Type A3-750, 3×150 mm×41 mm).

The set-up further comprised a cooler, a phase separator 6, a lean air inlet, vessels and devices for reactant metering, vessels and devices for the heteroazeotrope, a receiver for the bottom discharge as well as a receiver for stabilization of the column.

First a stabilized reaction mixture was produced by a discontinuous experiment which essentially consisted of 2-octanol, acrylic acid and also 2-octyl acrylate and methanesulfonic acid catalyst. The starting materials for producing this reaction mixture were used in the ratios which corresponded to the starting mixture metered in later. The starting mixture for producing the reaction mixture comprised 45% by weight 2-octanol, 25% by weight acrylic acid pure, 0.93% by weight 100% methanesulfonic acid, 29% by weight cyclohexane, 0.04% by weight calculated as 100% hypophosphorous acid, 0.1% by weight phenothiazine and 0.002% by weight CuCl. The production was carried out analogous to example 2.

The bath temperature was set to 128° C., that of the cover to 95° C.

In total, 667 g/h starting mixture was metered into the first reactor 1. The starting mixture may be initially charged in one or more vessels, wherein the substance mixtures in the vessels may differ in their composition. In total, the starting mixture consisted of 45% by weight 2-octanol, 25% by weight acrylic acid pure, 0.93% by weight 100% methanesulfonic acid, 29% by weight cyclohexane, 0.04% by weight hypophosphorous acid (calc. 100%) and 0.1% by weight phenothiazine. In addition, CuCl was added as a solid at regular intervals such that the average concentration was 0.002% by weight.

350 g/h of cyclohexane were also metered in, stabilized with HO-TEMPO, to the top of the column. Level-controlled material was continuously removed from reactor 1. The reactor content (1) was regularly analyzed by gas chromatography.

When equilibrium and a constant bottom temperature of 108° C. had been reached, the reaction discharge vessel was changed, the material of the forerun was discarded and the new reaction discharge collected.

In a second experiment, this material was fed to reactor 1, which was still filled with reaction mixture from the previous experiment, at the same rate as it was removed in the first experiment. In addition, 150 g/h of cyclohexane were added to the bottoms and 50 g/h of cyclohexane (stabilized with HO-TEMPO) were added to the top of the column. Again, level-controlled material was continuously removed from reactor 1.

When equilibrium and a constant bottom temperature of 113° C. had been reached, the reaction discharge vessel was changed, the material of the forerun was discarded and the new reaction discharge collected.

In a third experiment, this material was fed to reactor 1, which was still filled with reaction mixture from the previous experiment, at the same rate as it was removed in the second experiment. In addition, 150 g/h of cyclohexane were added to the bottoms and 50 g/h of cyclohexane (stabilized with HO-TEMPO) were added to the top of the column. Again, level-controlled material was continuously removed from reactor 1.

When equilibrium and a constant bottom temperature of 114° C. had been reached, the reaction discharge vessel was changed, the material of the forerun was discarded and the new reaction discharge collected.

This material was investigated by acid-base titration and qualitative and quantitative gas chromatography. Here, octyl acrylate, cyclohexane and 2-octanol were quantified by gas chromatography whereas acrylic acid and methanesulfonic acid were determined by acid-base titration. The content of the secondary components octenes, diacrylic acid esters and oxyesters was determined by qualitative GC. In the case of the secondary components which are only present at a low fraction in the reaction mixture, it was further assumed that the values determined as area percentages correspond to the values that would be obtained by quantification.

This reaction discharge comprised 67.6% by weight 2-octyl acrylate, 22.4% by weight cyclohexane, 3.1% by weight 2-octanol, 2.4% by weight acrylic acid, 1.1% by weight methanesulfonic acid, 0.26% by weight octenes, 1.1% by weight diacrylic acid ester and 1.5% by weight oxyester.

The reaction discharge was subjected to a continuous neutralization in an alkaline extraction unit 7 and then a waterwash in a waterwash extraction unit 8.

The alkaline extraction unit 7 comprises a continuous apparatus for neutralization with 10% aqueous sodium hydroxide solution NaOH. This continuous apparatus consists of a mixing pump and a phase separator 6 having a diameter of 40 mm and a phase interface regulator.

At a temperature of 30° C., 0.76 kg/h of the diluted aqueous sodium hydroxide solution NaOH and 3.3 kg/h of the reaction discharge were dispersed by the mixing pump and separated in the phase separator 6.

The neutralized organic phase was then dispersed in the waterwash extraction unit 8 with 5 kg/h of water by a static mixer (Kenics-Mischer (https://de.wikipedia.org/wiki/Statischer_Mischer, retrieved on 11.23.2021) having a 4.9 mm internal diameter and 27 elements) and subsequently separated in a phase separator 6 having a diameter of DN40.

### Example 4: Distillation (Process Stages IV to VII)

The distillative purification of 2-octyl acrylate was carried out in four successive, continuously operated process stages.
Cyclohexane separation (IV)
2-Octanol separation (V)
Pure boiler separation (VI)
High-boiler separation (VII)

The experimental setup was identical for each individual process stage IV to VII and comprised in each case a thin-layer evaporator having a surface area of 0.016 square meters, which was used in the respective rectification column 9, 10, 11, 12. The respective rectification column 9, 10, 11, 12 had a diameter of 30 mm and a packing length of 100 cm. The packing in the respective rectification column 9, 10, 11, 12 was Montz A3-750. The respective condenser 5 belonging to the rectification column 9, 10, 11, 12 with reflux divider for the reflux and the distillate discharge was of course present, as well as the vessels and pumps for the metered addition of the reactants, the vessels and devices for the distillate, the vessel for the bottom discharge and the vessel for stabilizing the column 9, 10, 11, 12.

The thin-layer evaporator was heated with Marlotherm oil.

The individual components were determined in this example 4 by various measurement methods. Water was determined by a Karl-Fischer titration, whereas 2-octyl acrylate, cyclohexane and 2-octanol were determined by quantitative GC. The residual components were determined by qualitative GC.

### Cyclohexane Separation

The mixture arising from the waterwash extraction unit 8 had the following composition:

| | |
|---|---|
| Water | 0.30% by weight |
| Cyclohexane | 23.80% by weight |
| Octenes | 0.29% by weight |
| 2-Octanol | 3.95% by weight |
| 2-Octyl acrylate | 68.80% by weight |
| Diacrylic acid ester | 1.14% by weight |
| Oxyester | 1.54% by weight |
| Unknown | Remainder |

The amount of feed to the head of the azeotrope rectification column 9 was 300 g/h.

The absolute pressure at the top of the column was 100 mbar and the bottom temperature was 105° C.

The bottom product running out at 230 g/h had the following composition:

| | |
|---|---|
| Water | 0.00% by weight |
| Cyclohexane | 1.07% by weight |
| Octenes | 0.34% by weight |
| 2-Octanol | 5.14% by weight |
| 2-Octyl acrylate | 89.72% by weight |
| Diacrylic acid ester | 1.49% by weight |
| Oxyester | 2.01% by weight |
| Unknown | Remainder |

The distillate running off at 70 g/h had the following composition:

| | |
|---|---|
| Water | 1.28% by weight |
| Cyclohexane | 98.50% by weight |
| Octenes | 0.11% by weight |
| 2-Octanol | 0.05% by weight |
| 2-Octyl acrylate | 0.06% by weight |
| Diacrylic acid ester | 0.00% by weight |
| Oxyester | 0.00% by weight |

### 2-Octanol Separation

For the separation of 2-octanol, the same experimental apparatus was used as in the previous process stage. The bottom runoff from the cyclohexane separation was metered at a rate of 230 g/h to the top of the 2-octanol rectification column 10. The absolute pressure at the top of the column was 10 mbar and the bottom temperature was 89° C.

The bottom product running out at 190 g/h had the following composition:

| | |
|---|---|
| Water | 0.00% by weight |
| Cyclohexane | 0.00% by weight |
| Octenes | 0.00% by weight |
| 2-Octanol | 0.01% by weight |
| 2-Octyl acrylate | 95.47% by weight |
| Diacrylic acid ester | 1.80% by weight |
| Oxyester | 2.43% by weight |
| Unknown | Remainder |

The distillate running off at 40 g/h had the following composition:

| | |
|---|---|
| Water | 0.00% by weight |
| Cyclohexane | 6.12% by weight |
| Octenes | 1.98% by weight |
| 2-Octanol | 29.50% by weight |
| 2-Octyl acrylate | 62.40% by weight |
| Diacrylic acid ester | 0.00% by weight |
| Oxyester | 0.00% by weight |

### Pure Boiler Separation

For the pure boiler separation, the same experimental apparatus was used as in the previous process stages. The bottom runoff obtained from the 2-octanol separation experiment was metered at a rate of 190 g/h to the bottom of the pure boiler rectification column 11. The absolute pressure at the top of the column was 10 mbar and the bottom temperature was 95° C. A reflux ratio of 3 g/g was set at the reflux divider. To avoid polymerization in the experimental apparatus, 2 g/h of 2-octyl acrylate (stabilized with 1% phenothiazine) was metered in at the top of the pure boiler rectification column 11.

The bottom product running out at 32 g/h had the following composition:

| | |
|---|---|
| Water | 0.00% by weight |
| Cyclohexane | 0.00% by weight |
| Octenes | 0.00% by weight |
| 2-Octanol | 0.00% by weight |
| 2-Octyl acrylate | 74.49% by weight |
| Diacrylic acid ester | 10.69% by weight |
| Oxyester | 14.44% by weight |
| Phenothiazine | 0.13% by weight |
| Unknown | Remainder |

The distillate running off at 160 g/h had the following composition:

| | |
|---|---|
| Water | 0.00% by weight |
| Cyclohexane | 0.00% by weight |
| Octenes | 0.00% by weight |
| 2-Octanol | 0.01% by weight |
| 2-Octyl acrylate | 99.70% by weight |
| Diacrylic acid ester | 0.00% by weight |
| Oxyester | 0.00% by weight |
| Unknown | Remainder |

### High-Boiler Separation

For the separation of the high-boilers, the same experimental apparatus was used as in the previous process stages. The bottom runoff obtained from the pure boiler separation was metered at a rate of 32 g/h to the bottom of the high-boiler rectification column 12. The absolute pressure at the top of the column was 5 mbar and the bottom temperature was 100° C. A reflux ratio of 3 g/g was set at the reflux divider.

The bottom product running out at 16 g/h had the following composition:

| | |
|---|---|
| Water | 0.00% by weight |
| Cyclohexane | 0.00% by weight |
| Octenes | 0.00% by weight |
| 2-Octanol | 0.00% by weight |
| 2-Octyl acrylate | 49.27% by weight |
| Diacrylic acid ester | 21.37% by weight |
| Oxyester | 28.87% by weight |
| Phenothiazine | 0.25% by weight |
| Unknown | Remainder |

The distillate running off at 16 g/h had the following composition:

| | |
|---|---|
| Water | 0.00% by weight |
| Cyclohexane | 0.00% by weight |
| Octenes | 0.00% by weight |
| 2-Octanol | 0.00% by weight |
| 2-Octyl acrylate | 99.72% by weight |
| Diacrylic acid ester | 0.00% by weight |
| Oxyester | 0.00% by weight |
| Unknown | Remainder |

1.-15. (canceled)

16. A process for producing 2-octyl (meth)acrylate by reacting 2-octanol with (meth)acrylic acid in the presence of an acidic esterification catalyst, a polymerization inhibitor and the azeotroping agent cyclohexane, comprising the steps of:
providing a reactor unit, wherein a reactor having a reactor heating element is located within the reactor unit,
feeding 2-octanol, (meth)acrylic acid, acidic esterification catalyst, cyclohexane and polymerization inhibitor into the reactor,
carrying out an esterification in the reactor to form a liquid reaction mixture, wherein the esterification in the reactor is conducted at a bottom temperature in the range of 90 to 130° C. and at an absolute pressure in the range of 0.5 to 2.0 bar, and a resulting reaction discharge from the reactor is obtained,
wherein the resulting reaction discharge comprises at least 2-octyl (meth)acrylate, 2-octanol, (meth)acrylic acid, acidic esterification catalyst, cyclohexane, water of esterification and polymerization inhibitor, and the water of esterification formed in the esterification together with the cyclohexane azeotroping agent forms a heterogeneous azeotrope,
evaporating the heterogeneous azeotrope from the liquid reaction mixture of the reactor, wherein the evaporation is accomplished by the reactor heating element, and
removing the gaseous heterogeneous azeotrope from the reactor,
wherein the gaseous heterogeneous azeotrope is condensed in a condenser and is then fed to a phase separator and the water of esterification is separated off as the lower phase and the cyclohexane as upper phase in this phase separator.

17. The process according to claim 16, wherein the gaseous heterogeneous azeotrope is fed to an azeotrope rectification column unit downstream of the reactor unit, wherein an azeotrope rectification column is located within the azeotrope rectification column unit, and the azeotrope rectification column is operated at an absolute pressure in the range of 0.5 to 2 bar and at a bottom temperature in the range of 90 to 130° C., and the heterogeneous azeotrope is removed via the top of the azeotrope rectification column, is condensed in a condenser and is then fed to a phase separator,
wherein the water of esterification is separated off as the lower phase in the phase separator, while the cyclohexane is separated off as the upper phase in the phase separator.

18. The process according to claim 16, wherein the cyclohexane obtained as upper phase in the phase separator is partially or fully recycled to the reactor unit.

19. The process according to claim 17, wherein the cyclohexane obtained as upper phase in the phase separator is recycled to the reactor unit at a proportion by weight in the range of 40% by weight to 100% by weight, and
the resulting residual proportion of the cyclohexane obtained is fed into the area from below the top to the middle of the azeotrope rectification column.

20. The process according to claim 16, wherein subsequent to the esterification, an alkaline extraction of the resulting reaction discharge is carried out by means of an alkaline solution,
in which the acidic esterification catalyst and unreacted (meth)acrylic acid are neutralized in a neutralization extraction unit, and
which results in an upper neutralization phase comprising 2-octyl (meth)acrylate, and a lower neutralization phase comprising salts generated by the neutralization and water.

21. The process according to claim 20, wherein subsequent to the alkaline extraction of the reaction discharge, the upper neutralization phase obtained from the neutralization extraction unit is extracted with water in a waterwash extraction unit,
wherein a lower waterwash phase comprising water and salt residues and an upper waterwash phase comprising 2-octyl (meth)acrylate are formed.

22. The process according to claim 20, wherein subsequent to the alkaline extraction of the reaction discharge or subsequent to the waterwash extraction, the azeotroping agent cyclohexane is separated off from the upper neutralization phase or from the upper waterwash phase in an azeotrope rectification column unit,
wherein an azeotrope rectification column is located within the azeotrope rectification column unit, and
the azeotrope rectification column is operated at a reduced absolute pressure in the range of 0.05 to 0.9 bar and at a bottom temperature in the range of 70 to 120° C., and an organic phase is removed via the top of the azeotrope rectification column comprising the cyclohexane azeotroping agent, octenes, 2-octanol and a mass flow proportion of 2-octyl (meth)acrylate in the range of 0.1 to 5.0%, based on the upper neutralization phase fed in or the upper waterwash phase fed in,
wherein the components fed in overhead form an azeotrope mass flow, this being condensed and fed into the reactor of the reactor unit in the range of 50 to 100%,
wherein the residual azeotrope mass flow proportion is condensed and is recycled to the top of the azeotrope rectification column, while high-boilers comprising di(meth)acrylic acid esters and oxy esters, such as the alkoxyalkyl esters of (meth)acrylic acid, and also 2-octanol and 2-octyl (meth)acrylate are removed through a bottom discharge of the azeotrope rectification column,
wherein in the range of 20 to 95% of the mass flow of the bottom discharge flows through an evaporator and is subsequently recycled to the azeotrope rectification column.

23. The process according to claim 20, wherein the 2-octyl (meth)acrylate is separated in a dividing wall column from the residual components comprising cyclohexane and 2-octanol.

24. The process according to claim 22, wherein subsequent to the removal of the cyclohexane azeotroping agent, 2-octanol is separated off from the mass flow produced by the bottom discharge of the azeotroping agent rectification column in a 2-octanol rectification column unit or a 2-octanol evaporation unit,
wherein a 2-octanol rectification column is located in the 2-octanol rectification column unit or a 2-octanol evaporator is located within the 2-octanol evaporation unit, and the 2-octanol rectification column or the 2-octanol evaporator is operated at a reduced absolute pressure in the range of 0.005 to 0.10 bar and at a bottom temperature in the range of 70 to 130° C., and the mass flow produced by the bottom discharge of the azeotrope rectification column is metered in to the area below the head to the middle of the 2-octanol rectification column or to a 2-octanol inlet of the 2-octanol evaporator, and
the components discharged via the head of the 2-octanol rectification column or via a 2-octanol evaporator outlet of the 2-octanol evaporator are fed to the reactor unit in the range of 20 to 50%, based on the mass flow of the discharged components,
wherein the discharged components in the range of 2 to 40% by weight consist of 2-octanol, while 2-octyl (meth)acrylate and high-boilers comprising di(meth)acrylic acid esters and oxy esters are withdrawn through a bottom discharge of the 2-octanol rectification column or the 2-octanol evaporator,
wherein 20 to 95% of the mass flow of the bottom discharge flows through an evaporator and is subsequently recycled to the 2-octanol rectification column or to the 2-octanol evaporator.

25. The process according to claim 24, wherein subsequent to the separation of the 2-octanol, a pure boiler separation from the mass flow provided through the bottom discharge of the 2-octanol rectification column or the 2-octanol evaporator is carried out in a pure boiler rectification column unit or a pure boiler evaporation unit,
wherein a pure boiler rectification column is located within the pure boiler rectification column unit or a pure boiler evaporator within the pure boiler evaporation unit, and
the pure boiler rectification column or the pure boiler evaporator is operated at a reduced absolute pressure in the range of 0.002 to 0.05 bar and at a bottom temperature in the range of 80 to 130° C., and
the mass flow produced by the bottom discharge of the 2-octanol rectification column or the 2-octanol evaporator is metered into the area from below the head to the middle of the pure boiler rectification column or to a pure boiler evaporator inlet of the pure boiler evaporator, and to stabilize the pure boiler rectification column or the pure boiler evaporator, 2-octyl (meth)acrylate and a polymerisation inhibitor, each in the range of 0.01 to 1.0%, based on the mass flow produced by the bottom discharge of the 2-octanol rectification column or by the bottom discharge of the 2-octanol evaporator, are metered into the area from the bottom up to the middle of the pure boiler rectification column or to a pure boiler evaporator inlet of the pure boiler evaporator, and
the 2-octyl (meth)acrylate is withdrawn via the head of the pure boiler rectification column or via a pure boiler evaporator outlet of the pure boiler evaporator, while high-boilers comprising di(meth)acrylic acid esters and oxy esters, such as the alkoxyalkyl esters of (meth)acrylic acid, are withdrawn through a bottom discharge of the pure boiler rectification column or of the pure boiler evaporator.

26. The process according to claim 25, wherein subsequent to the pure boiler separation, a high-boiler separation from the mass flow produced through the bottom discharge of the pure boiler rectification column or of the pure boiler evaporator is carried out in a high-boiler rectification column unit or a high-boiler evaporation unit,
wherein a high-boiler rectification column is located within the high-boiler rectification column unit or a high-boiler evaporator within the high-boiler evaporator unit, and
the high-boiler rectification column or the high-boiler evaporator is operated at a reduced absolute pressure in the range of 0.002 to 0.05 bar and at a bottom temperature in the range of 80 to 150° C., and
the mass flow produced by the bottom discharge of the pure boiler rectification column, or the pure boiler evaporator is metered into the region from the bottom up to the middle of the high-boiler rectification column or to a high-boiler evaporator inlet of the high-boiler evaporator, and
the takeoff withdrawn via the top of the high-boiler rectification column, or the high-boiler evaporator is condensed and is fed into the region from the bottom up to the middle of the pure boiler rectification column or to the pure boiler evaporator inlet of the pure boiler evaporator.

27. The process according to claim 16, wherein the bottom temperature in the reactor of the reactor unit is in the range of 100 to 125° C.

28. The process according to claim 16, wherein cyclohexane is fed to the reactor of the reactor unit in an amount in the range of 100 to 600% by weight, based on the amounts by mass of 2-octanol and (meth)acrylic acid fed to the reactor of the reactor unit.

29. The process according to claim 16, wherein the proportion of catalyst in the reactor of the reactor unit is at maximum 10% by weight, based on the sum of the components of 2-octanol and (meth)acrylic acid present in the reactor of the reactor unit.

30. The process according to claim 16, wherein the components flowing into the reactor of the reactor unit without the azeotroping agent cyclohexane have the following proportions by weight:

| | |
|---|---|
| 2-octanol: | 40.00 to 84.39% by weight |
| (meth)acrylic acid: | 15.00 to 59.39% by weight |
| acidic esterification catalyst: | 0.50 to 10.00% by weight |
| polymerization inhibitor: | 0.01 to 1.00% by weight |
| residual components: | 0.10 to 5.00% by weight |

and the azeotroping agent cyclohexane is added in an amount such that a concentration of cyclohexane forms in the reactor of the reactor unit in the range of 10 to 90% by weight,
wherein the figures of the proportion by weight relate to the components present in the reactor of the reactor unit including the cyclohexane,
and/or without the azeotroping agent cyclohexane and without the water of esterification, the total components flowing out of the reactor of the reactor unit, namely the resulting liquid reaction discharge plus the portion evaporated from the reaction mixture and discharged from the reactor unit, have the following proportions by weight:

| | |
|---|---|
| 2-octyl (meth)acrylate: | 50.00 to 95.00% by weight |
| 2-octanol: | 1.00 to 30.00% by weight |
| (meth)acrylic acid: | 1.00 to 15.00% by weight |
| acidic esterification catalyst: | 0.50 to 10.00% by weight |
| polymerization inhibitor: | 0.01 to 1.00% by weight |
| residual components: | 2.49 to 10.00% by weight |

and the heterogeneous azeotrope formed by the cyclohexane and the water of esterification flows out of the reactor of the reactor unit at a concentration in the range of 10 to 50% by weight, where these figures of the percentage by weight refer to the total components flowing out including the cyclohexane and the water of esterification.
